(19) United States
(12) Patent Application Publication
(10) Pub. No.: US 2013/0280368 A1
(43) Pub. Date: Oct. 24, 2013

(54) METHOD FOR SCREENING FOR DIET PROVIDING PRODUCTION OF MILK HAVING IMMUNOREGULATORY ACTION

(71) Applicants: Hirohisa Izumi, Zama-shi (JP); Takashi Shimizu, Zama-shi (JP); Kazunori Sekine, Zama-shi (JP)

(72) Inventors: Hirohisa Izumi, Zama-shi (JP); Takashi Shimizu, Zama-shi (JP); Kazunori Sekine, Zama-shi (JP)

(73) Assignee: MORINAGA MILK INDUSTRY CO., LTD., Minato-ku, Tokyo (JP)

(21) Appl. No.: 13/809,877

(22) PCT Filed: Jan. 11, 2012
(86) PCT No.: PCT/JP2012/050312

§ 371 Date: Jan. 11, 2013

(30) Foreign Application Priority Data
Jan. 12, 2011 (JP) 2011-004090

(51) Int. Cl. A23C 9/20; C12N 15/10

(52) U.S. Cl. CPC A23C 9/20; C12N 15/1072
USPC 426/2; 435/6.13; 506/9

(57) ABSTRACT

A diet or a substance providing production of breast milk having an immunoregulatory action is screened for by identifying a diet or a substance that increases or decreases amount of mRNA present in milk of a mammal using correlation of mRNA profile in the milk and a diet ingested by the mammal or a substance contained in the diet as an index.

### TECHNICAL FIELD

The present invention relates to a method for screening for a diet providing production of milk having immunoregulatory action, and use thereof. They are useful in the fields of food, feed, and so forth.

### BACKGROUND ART

Exosomes are microvesicles secreted by living body cells or cultured cells, and it is known that miRNAs (microRNAs) and proteins exist therein. As body fluids in which presence of exosomes is confirmed, there are known blood (serum and plasma), breast milk, bronchoalveolar lavage fluid, malignant ascite fluid, synovial fluid, urine, amniotic fluid, sperm, saliva, and so forth. Among these body fluids, only blood serum has been investigated about RNAs so far, and all of the target RNAs are miRNAs. On the other hand, in the researches using cells other than body fluid such as cultured cells, RNAs in microvesicles derived from mast cells and glioblastoma have been investigated, and mRNAs are also investigated in these researches (Non-patent document 1 for all the technical background described in this paragraph).

As for milk, it has been reported that miRNAs are contained in bovine milk, bovine serum, and commercial powdered infant formula (Non-patent document 2), and it has been reported that miRNAs are contained in breast milk and various body fluids other than breast milk (Non-patent document 3). Further, it has been reported that miRNAs in breast milk are involved in immunoregulation (Non-patent document 4).

Furthermore, it has also been reported that miRNAs and mRNAs are contained in microvesicles derived from bovine milk (Non-patent document 5). However, mRNAs investigated in this report are only those coding for 6 kinds of proteins, α-S1-casein, α-S2-casein, β-casein, κ-casein, β-lactoglobulin, and MFG-E8, which are milk proteins, and EF-1α, which is a transcription factor.

miRNAs and mRNAs derived from exosomes in human saliva have recently been investigated, and 509 kinds of mRNAs have been detected by using a microarray (Non-patent document 6). Further, potentiality as a biomarker of renal diseases has been suggested for nucleic acids derived from exosomes in human urine, and these nucleic acids include mRNAs (Non-patent document 7).

Breast milk contains secretory IgA, lactoferrin, lysozyme, cytokines, and so forth, and it is considered that it protects infants from infection, and promotes development of infant's immunity (Non-patent document 8). Actually, it is known that children grown up on breast milk involve a lower risk of infection in the bronchi or intestinal tract compared with children not grown in such a manner. Breast milk contains IgA, lactoferrin, glycoproteins, glycolipids etc., which show antibacterial activities, as well as cytokines, which regulate immunocytes.

As mentioned above, it has been reported that miRNAs involved in immunoregulation exist in milk, and that mRNAs coding for milk proteins and transcription factor are contained in microvesicles derived from bovine milk, but it is not known that mRNAs involved in immunoregulation exist in milk.

### PRIOR ART REFERENCES

### Non-Patent Documents

Non-patent document 1: Simpson R. J., Expert Rev. Proteomics, 6:267-283, 2009
Non-patent Document 2: Chen, X. et al., Cell Res., 20:1128-1137, 2010
Non-patent Document 3: Weber, J. A. et al., Clin. Chem., 56:1733-1741, 2010
Non-patent document 4: Kosaka, N. et al., Silence, 1:7, 2010
Non-patent document 5: Hata, T. et al., Biochem. Biophys. Res. Commun., 396:528-533, 2010
Non-patent document 6: Palanisamy, V. et al., PLoS One, 3(9):e3148, 2008
Non-patent document 7: Miranda. K. C. et al., Kidney Int., 78:191-199, 2010
Non-patent document 8: Goldman, A. S., Breastfeed. Med., 2(4):195-204, 2007

### SUMMARY OF THE INVENTION

### Object to be Achieved by the Invention

An object of the present invention is to provide a method for screening for a diet providing production of milk having an immunoregulatory action, a food having an immunoregulatory action, and a method for producing it.

### Means for Achieving the Object

The inventors of the present invention conducted researches with paying attention to the fact that breast milk affected maturation of infant's immune system. As a result, they found that mRNAs of immunity-related genes were contained in breast milk, and accomplished the present invention.

The present invention thus provides a method for screening for a diet or a substance providing production of breast milk having an immunoregulatory action, which comprises identifying a diet or a substance that increases or decreases amount of mRNA present in milk of a mammal by using correlation of mRNA profile in the milk and a diet ingested by the mammal or a substance contained in the diet as an index.

In an embodiment of the aforementioned method, the immunoregulatory action is an immunostimulating action, and when either one or both of the following conditions are satisfied, it is determined that the diet or the substance provides production of breast milk having an immunostimulating action:

(A) the mRNA is a mRNA of which amount in milk of a rat increases when a Bifidobacterium is orally administered to the rat, and ingestion of the diet increases amount of the mRNA,

(B) the mRNA is a mRNA of which amount in milk of a rat decreases when a Bifidobacterium is orally administered to the rat, and ingestion of the diet decreases amount of the mRNA.

In another embodiment of the aforementioned method, the immunoregulatory action is an immunosuppressive action, and when either one or both of the following conditions are satisfied, it is determined that the diet or the substance provides production of breast milk having an immunosuppressive action:

(a) the mRNA is a mRNA of which amount in milk of a rat increases when a Bifidobacterium is orally administered to the rat, and ingestion of the diet decreases amount of the mRNA,

(b) the mRNA is a mRNA of which amount in milk of a rat decreases when a Bifidobacterium is orally administered to the rat, and ingestion of the diet increases amount of the mRNA.

In an embodiment of the aforementioned method, mRNA profiles in the milk observed before and after ingestion of the diet are compared, and when amount of at least one kind of mRNA in the milk observed after the ingestion is higher than that observed before the ingestion, it is determined that the diet increases the amount of the mRNA in the milk.

In a preferred embodiment of the aforementioned method, mRNA profiles in the milk observed before and after ingestion of the diet are compared, and when amount of a mRNA in the milk observed after the ingestion is 1.5 times or more of that observed before the ingestion, it is determined that the diet increases the amount of the mRNA in the milk.

In another embodiment of the aforementioned method, mRNA profiles in the milk observed before and after ingestion of the diet are compared, and when amount of at least one kind of mRNA in the milk observed after the ingestion is lower than that observed before the ingestion, it is determined that the diet decreases the amount of the mRNA in the milk.

In a preferred embodiment of the aforementioned method, mRNA profiles in the milk observed before and after ingestion of the diet are compared, and when amount of a mRNA observed after the ingestion is 0.67 times or less of that observed before the ingestion, it is determined that the diet decreases the amount of the mRNA in the milk.

Further, in a preferred embodiment of the aforementioned method, the mammal is a human, a rat, or a bovine.

In a preferred embodiment of the aforementioned method, the mRNA profiles comprises amount of mRNA selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl211, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp2l1, Nr3c2, Klf4, Dhx8, Myh14, Mcam, Dusp3, Acer2, L3 mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4bp1, Naaa, Expi, Fgf7, RGD1562492, Lama2, Ahnak, Sec3l1, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdspl, Tp53bp1, Fam60a, Pik3r2, RGD1305089, Arhgef5, Nek7, Rpt, Vhl, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, Klf11, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4 m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnrpll, LOC687994, Sptbn1, Dtx31, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3cl1, LOC100302372, RGD1311892, Fbxo42, Daam1, K1h15, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, Slc25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, Nolc1, Sult1a1, Ankrd17, Trim2, Nfib, Cd14, S100a10, LOC687105, Ccdc123, RGD1309537, Pols, Aplf, Gulp1, Slc40a1, Tfdp2, Dyrk2, Anxa5, Nr3c1, RGD1306862, Rkhd2, Cdc42 bpb, Numa1, Mocos, Mafb, Akap11, Diaph1, Znrf1, Sos1, Rhob, Pkp2, Srf, RGD1310159, Cdh1, Rnf19a, Pdlim1, Csnk1g3, Hectd1, Taf5l, Noc3l, Nfat5, RGD1562529, Atl3, Ptpn9, Nedd4l, Wipi1, Gnal, Tle1, Pprc1, Src, Clic1, Secisbp21, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc215, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, Klhl24, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbxl8, Chd1, Tp53 bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rpl8, Prdx1, Phrf1, Pcm1, Mapk12, Slc1a4, Prl3d4, Trim16, LOC689842, Cacna1e, RT1-EC2, Gal, Arnt2, Stk11ip, Sdc3, RGD1563155, Tp53i11, Mpz, Dnmt31, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Ttl, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb3l1, K1k12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fhl3, Olr515, Tpsab1, RGD1564961, Hdhd2, Farp1, Ptprs, Hand1, Rpl37a, Tcf15, Neurod2, Usp20, Foxo3, Aldh1l2, RGD1308706, Nov, RGD1566130, Slc34a2, Gzma, Hist1h4b, Iqgap3, Enpp2, Tm7sf2, Exo1, Slc9a1, Rpl37, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, Slc25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad54l, Vps37c, Hmga1, Olr1572, Furin, RT1-CE5, Rab15, Folr1, Fundc1, Pak3, Aqp2, Hapln2, Tmem37, Gjd3, Lamb1, Cox5a, Cfd, Jak3, Magt1, Sc4mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Msl3l2, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bcan, Foxc2, Jph1, Ccl27, Pdia3, Gabrq, Surf6, Runx3, Abcb6, Cdk2, LOC687813, and Porcn.

Further, in a preferred embodiment of the aforementioned method, the mRNA of which amount in milk of a rat increases when a Bifidobacterium is orally administered to the rat is selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl2l1, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp2l1, Nr3c2, Klf4, Dhx8, Myh14, Mcam, Dusp3, Acer2, L3mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4 bp1, Naaa, Expi, Fgf7, RGD1562492, Lama2, Ahnak, Sec3l1, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdsp1, Tp53bp1, Fam60a, Pik3r2, RGD1305089, Arhgef5, Nek7, Rp2, Vhl, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnrpll, LOC687994, Sptbn1, Dtx31, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3cl1, LOC100302372, RGD1311892, Fbxo42, Daam1, Klhl5, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, Slc25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, Nolc1, Sult1a1, Ankrd17, Trim2, Nfib, Cd14, S100a10, LOC687105, Ccdc123, RGD1309537, Pols, Aplf, Gulp1, Slc40a1, Tfdp2, Dyrk2, Anxa5, Nr3c1, RGD1306862, Rkhd2, Cdc42bpb, Numa1, Mocos, Mafb, Akap11, Diaph1, Znrf1, Sos1, Rhob, Pkp2, Srf, RGD1310159, Cdh1, Rnf19a, Pdlim1, Csnk1g3, Hectd1, Taf5l, Noc3l, Nfat5, RGD1562529, Atl3, Ptpn9, Nedd4l, Wipi1, Gnal, Tle1, Pprc1, Src, Clic1, Secisbp21, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc215, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, Klhl24, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbx18, Chd1, Tp53 bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rpl8, Prdx1, Phrf1, Pcm1, and Mapk12

The aforementioned mRNAs are mRNAs corresponding to the genes mentioned in Tables 1 to 14 mentioned later. In the tables, the gene names mentioned in the columns of “Gene Symbol” and “Entrez Gene ID” are the gene names used in the Entrez Gene database of NCBI (National Center for Biotechnology Information), and the gene names mentioned in the column of “Gene Name” are the generic gene names. The same shall apply to Tables 15 to 22 mentioned later.

Further, in a preferred embodiment of the aforementioned method, the mRNA of which amount in milk of a rat decreases when a Bifidobacterium is orally administered to the rat is selected from the group consisting of Slc1a4, Prl3d4, Trim16, LOC689842, Cacna1e, RT1-EC2, Gal, Arnt2, Stk11ip, Sdc3, RGD1563155, Tp53i11, Mpz, Dnmt31, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Tt1, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb3l1, K1k12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fh13, Olr515, Tpsab1, RGD1564961, Hdhd2, Farp1, Ptprs, Hand1, Rp137a, Tcf15, Neurod2, Usp20, Foxo3, Aldh1l2, RGD1308706, Nov, RGD1566130, Slc34a2, Gzma, Hist1h4b, Iqgap3, Enpp2, Tm7sf2, Exo1, Slc9a1, Rp137, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, Slc25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad54l, Vps37c, Hmga1, Olr1572, Furin, RT1-CE5, Rab15, Folr1, Fundc1, Pak3, Aqp2, Hapln2, Tmem37, Gjd3, Lamb1, Cox5a, Cfd, Jak3, Magt1, Sc4mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Msl3l2, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bcan, Foxc2, Jph1, Cc127, Pdia3, Gabrq, Surf6, Runx3, Abcb6, Cdk2, LOC687813, and Porcn.

The aforementioned mRNAs are mRNAs corresponding to the genes mentioned in Tables 15 to 22 mentioned later.

The present invention also provides a method for producing milk or a dairy product having an immunoregulatory action, which comprises the step of giving a diet or a substance identified to increase or decrease amount of mRNA in milk of a mammal by the aforementioned screening method to a mammal (except for human), and the step of collecting milk of the mammal.

In an embodiment of the aforementioned method, the immunoregulatory action is an immunostimulating action, and the diet or the substance is one determined to provide production of breast milk having an immunostimulating action.

In another embodiment of the aforementioned method, the immunoregulatory action is an immunosuppressive action, and the diet or the substance is one determined to provide production of breast milk having an immunosuppressive action.

The present invention also provides a method for producing a composition for oral ingestion having an immunostimulating action, which comprises adding a mRNA of which amount in milk of a rat increases when a Bifidobacterium is orally administered to the rat to a base for a composition for oral ingestion.

In a preferred embodiment of the aforementioned method, the mRNA is selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl2l1, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp2l1, Nr3c2, Klf4, Dhx8, Myh14, Mcam, Dusp3, Acer2, L3 mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4bp1, Naaa, Expi, Fgf7, RGD1562492, Lama2, Ahnak, Sec311, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdsp1, Tp53bp1, Fam60a, Pik3r2, RGD1305089, Arhgef5, Nek7, Rp2, Vhl, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, Klf11, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnrpll, LOC687994, Sptbn1, Dtx31, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3c11, LOC100302372, RGD1311892, Fbxo42, Daam1, Klhl5, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, Slc25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, Nolc1, Sult1a1, Ankrd17, Trim2, Nfib, Cd14, S100a10, LOC687105, Ccdc123, RGD1309537, Pols, Aplf, Gulp1, Slc40a1, Tfdp2, Dyrk2, Anxa5, Nr3c1, RGD1306862, Rkhd2, Cdc42bpb, Numa1, Mocos, Mafb, Akap11, Diaph1, Znrf1, Sos1, Rhob, Pkp2, Srf, RGD1310159, Cdh1, Rnf19a, Pdlim1, Csnk1g3, Hectd1, Taf51, Noc31, Nfat5, RGD1562529, Atl3, Ptpn9, Nedd41, Wipi1, Gnal, Tle1, Pprc1, Src, Clic1, Secisbp2l, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc2l5, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, Klhl24, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbxl8, Chd1, Tp53bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rp18, Prdx1, Phrf1, Pcm1, and Mapk12.

The present invention also provides a method for producing a composition for oral ingestion having an immunosuppressive action, which comprises adding a mRNA of which amount in milk of a rat decreases when a Bifidobacterium is orally administered to the rat to a base for a composition for oral ingestion.

In a preferred embodiment of the aforementioned method, the mRNA is selected from the group consisting of Slc1a4, Prl3d4, Trim16, LOC689842, Cacna1e, RT1-EC2, Gal, Arnt2, Stk11ip, Sdc3, RGD1563155, Tp53i11, Mpz, Dnmt3l, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Ttl, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb3l1, Klk12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fhl3, Olr515, Tpsab1, RGD1564961, Hdhd2, Farp1, Ptprs, Hand1, Rp137a, Tcf15, Neurod2, Usp20, Foxo3, Aldh1l2, RGD1308706, Nov, RGD1566130, Slc34a2, Gzma, Hist1h4b, Iqgap3, Enpp2, Tm7sf2, Exo1, Slc9a1, Rp137, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, Slc25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad541, Vps37c, Hmga1, Olr1572, Furin, RT1-CE5, Rab15, Folr1, Fundc1, Pak3, Aqp2, Hapln2, Tmem37, Gjd3, Lamb1, Cox5a, Cfd, Jak3, Magt1, Sc4mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Msl3l2, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bcan, Foxc2, Jph1, Ccl27, Pdia3, Gabrq, Surf6, Runx3, Abcb6, Cdk2, LOC687813, and Porcn.

In a preferred embodiment of the method for producing a composition for oral ingestion, the composition is a food for infants or a food for little children.

In a preferred embodiment of the method for producing a composition for oral ingestion, the food for infants or the food for little children is infant formula or follow-on formula.

### MODES FOR CARRYING OUT THE INVENTION

The method of the present invention is a method for screening for a diet or a substance providing production of breast milk having an immunoregulatory action, which comprises identifying a diet or a substance that increases or decreases amount of mRNA present in milk of a mammal by using correlation of mRNA profile in the milk and a diet ingested by the mammal or a substance contained in the diet as an index.

In an embodiment of the aforementioned method, the immunoregulatory action is an immunostimulating action, and when either one or both of the following conditions are satisfied, it is determined that the diet or the substance provides production of breast milk having an immunostimulating action:

(A) the mRNA is a mRNA of which amount in milk of a rat increases when a Bifidobacterium is orally administered to the rat, and ingestion of the diet increases amount of the mRNA,

(B) the mRNA is a mRNA of which amount in milk of a rat decreases when a Bifidobacterium is orally administered to the rat, and ingestion of the diet decreases amount of the mRNA.

In another embodiment of the aforementioned method, the immunoregulatory action is an immunosuppressive action, and when either one or both of the following conditions are satisfied, it is determined that the diet or the substance provides production of breast milk having an immunosuppressive action:

(a) the mRNA is a mRNA of which amount in milk of a rat increases when a Bifidobacterium is orally administered to the rat, and ingestion of the diet decreases amount of the mRNA,

(b) the mRNA is a mRNA of which amount in milk of a rat decreases when a Bifidobacterium is orally administered to the rat, and ingestion of the diet increases amount of the mRNA.

The present invention is based on a concept that an immunoregulatory action is expected to be obtained by oral administration of mRNA, because of the novel finding that mRNAs relating to immunoregulation are contained in milk, and the fact that breast milk promotes development of immunity in infants ingesting the breast milk (for example, Breastfeed Med., 2(4):195-204, 2007). And, on the basis of a prediction that mRNA profile in milk is affected by diet, it was thought to identify a diet or an active ingredient contained in it that could increase or decrease amount of mRNA present in milk.

The immunoregulatory action defined for the screening method, milk, dairy product, and so forth of the present invention includes, for example, both an action of enhancing immunopotentiating action, which functions for the purpose of “defense” against external attacks (immunostimulating action), and an immunosuppressive action suppressively functioning against overresponse by the immunity, i.e., allergic responses, autoimmune diseases, chronic inflammations etc., in which “hyperimmunoreaction” adversely affect living organisms.

The terms “immunostimulating action” and “immunosuppressive action” are used in a relative meaning. When an immunopotentiating action usually observed for breast milk of a certain mammal is enhanced after ingestion of the diet or the substance, the breast milk has an immunostimulating action, and when the immunopotentiating action is decreased after ingestion of the diet or the substance, the breast milk has an immunosuppressive action. When the immunopotentiating action observed after ingestion of the diet or the substance by a mammal is enhanced compared with that observed before the ingestion, the breast milk of the mammal has an immunostimulating action, and when the immunopotentiating action is decreased compared with that observed before the ingestion, the breast milk has an immunosuppressive action.

The correlation of mRNA profile in milk of a mammal and a diet ingested by the mammal or a substance contained in the diet can be investigated, for example, as follows.

Milk is collected from a mammal that ingested a diet, and mRNA profile in the milk is examined.

The mammal is not particularly limited, and examples include human, bovine, water buffalo, goat, ovine, swine, ape, dog, cat, rat, mouse, hamster, guinea pig, and so forth. The mammal is preferably human or bovine.

In the present invention, the mRNA profile consists of type and amount of mRNA. The mRNA may consist of one kind of mRNA, or arbitrary two or more kinds of mRNAs. Type of mRNA is not particularly limited, so long as those existing in milk and involved in immunoregulation are chosen. As described above, it is known that breast milk promotes development of immunity in infants who ingest it (for example, Breastfeed Med., 2(4):195-204, 2007). Therefore, it is suggested that mRNAs contained in milk include those involved in immune functions. Further, as shown in the examples, it has been demonstrated that mRNAs contained in milk include those of which amount is increased by administration of a Bifidobacterium having an immunoregulatory action, and those of which amount is decreased by the same. This fact means that mRNAs contained in milk include those functioning in an immunostimulating manner, and those functioning in an immunosuppressive manner.

Type of mRNA is not particularly limited, so long as those existing in milk and involved in immunoregulation are chosen. Specific examples include, for example, mRNA of which amount in milk of a rat increases when a Bifidobacterium is orally administered to the rat, and mRNA of which amount in milk of a rat decreases when a Bifidobacterium is orally administered to the rat.

Examples of mRNA of which amount in milk of a rat increases when a Bifidobacterium is orally administered to the rat include those mentioned in Tables 1 to 14 mentioned later.

Examples of mRNA of which amount in milk of a rat decreases when a Bifidobacterium is orally administered to the rat include those mentioned in Tables 15 to 22 mentioned later.

In this specification, mRNA is specified by a name of corresponding gene, i.e., the gene used as a template for transcription of the mRNA.

A certain gene and a protein encoded by it may have different sequences depending on type of organism or due to individual difference. Genes showing high sequence homology and coding for proteins having the same functions or analogous functions are often regarded as the same genes or analogous genes as homologues or orthologues. In the present invention, mRNAs derived from the same or analogous genes are regarded as the same mRNAs. Therefore, even when “mRNA” in the expression “mRNA profile in milk of a mammal” and corresponding “mRNA” of which amount in milk of a rat increases or decreases when a Bifidobacterium is orally administered to the rat have different sequences, they may be regarded as the same mRNA, so long as they show high sequence homology and code for proteins having the same or analogous functions.

mRNA is not limited to mRNA having a known sequence, but mRNA may contain substitution, deletion, insertion, addition, or inversion of one or several nucleotides, so long as the encoded protein has function the same as or analogous to that of a protein encoded by a known sequence. Specific examples of such mRNA include, for example, mRNA having a nucleotide sequence showing a homology of 80% or more, preferably 90% or more, more preferably 95% or more, still more preferably 98% or more, to a known mRNA.

The amount of mRNA may be an absolute amount or a relative amount. The relative amount may be a relative amount based on an average amount in a certain mammal, or may be a relative amount observed after ingestion of a diet based on the amount observed before the ingestion. For the measurement of the amount of mRNA, methods usually used for measurement of mRNA amount such as quantitative reverse transcription PCR (qRT-PCR) can be employed. The amount of mRNA can also be measured by the microarray method. As for extraction of mRNA from milk, methods usually used for extraction of mRNA can be employed, and a commercially available mRNA isolation kit can also be used.

Further, amount of mRNA present in milk can also be indirectly measured by measuring expression amount of the mRNA in mammary glandular cells.

Correlation of mRNA profile in milk of a mammal and a diet ingested by the mammal or a substance contained in the diet is examined. The correlation of the mRNA profile in milk of a mammal and a diet ingested by the mammal or a substance contained in the diet refers to correlation of the mRNA profile and presence or absence of the diet or the substance or amount of the diet or the substance. For example, if amounts of one or more kinds of mRNAs in milk of a mammal which has ingested a certain diet or substance are higher or lower than those observed in the mammal which has not ingested the diet or the substance, the diet or the substance and the mRNA profile have positive or negative correlation, respectively. Further, also when amounts of one or more kinds of mRNAs in milk of a mammal which has ingested a certain diet or substance are higher than those observed in the mammal which has not ingested the diet or the substance, and at the same time, amounts of other one or more kinds of mRNAs in milk of a mammal which has ingested a certain diet or substance are lower than those observed in the mammal which has not ingested the diet or the substance, the diet or the substance and the mRNA profile have correlation. Further, if ingestion of a certain diet or substance does not affect mRNA profile, the substance and the mRNA profile do not correlate with each other.

Specifically, for example, when mRNA profiles in the milk observed before and after ingestion of the diet are compared, and amount of a certain kind of mRNA observed after the ingestion is higher than that observed before the ingestion, it is determined that the diet increases the amount of the mRNA in the milk. The degree of increase in amount of mRNA in the milk observed after the ingestion of the diet is, for example, 1.5 times or more, preferably 2 times or more, more preferably 5 times or more, still more preferably 10 times or more, of that observed before the ingestion.

Further, when mRNA profiles in the milk observed before and after ingestion of the diet are compared, and amount of a certain kind of mRNA observed after the ingestion is lower than that observed before the ingestion, it is determined that the diet decreases the amount of the mRNA in the milk. The degree of decrease in amount of mRNA in the milk observed after the ingestion of the diet is, for example, 0.67 times or less, preferably 0.5 times or less, more preferably 0.2 times or less, still more preferably 0.1 times or less, of that observed before the ingestion.

Furthermore, measurement of mRNA profile before ingestion of a diet is not indispensable, and correlation of a diet and amount of mRNA can also be examined by comparing mRNA profile measured after ingestion of a diet with ordinary mRNA profile of an objective mammal measured beforehand.

mRNA of which correlation is investigated may consist of a single kind of mRNA, or arbitrary two or more kinds of mRNAs.

In another embodiment, mRNA profile in milk and mRNA profile in serum or plasma are compared, and if amount of mRNA contained in both of milk and serum or plasma is increased by ingestion of the diet at a higher degree in milk compared with that observed in serum or plasma, it is determined that the diet increases amount of the mRNA present in milk. The degree of increase in amount of mRNA in milk is, for example, 1.5 times or more, preferably 2 times or more, more preferably 5 times or more, still more preferably 10 times or more, of that observed in serum or plasma.

Further, when mRNA profile in milk and mRNA profile in serum or plasma are compared, if amount of a mRNA contained in both of milk and serum or plasma is decreased by ingestion of the diet at a larger degree in milk compared with that observed in serum or plasma, it is determined that the diet decreases amount of the mRNA present in milk. The degree of decrease in amount of mRNA in milk compared with that in serum or plasma is, for example, 1.5 times or more, preferably 2 times or more, more preferably 5 times or more, still more preferably 10 times or more. For example, when amount of a certain mRNA decreases to 0.5 times in serum or plasma and decreases to 0.3 times in milk, degree of the decrease in milk is 1.67 (0.5/0.3) times higher than that in serum or plasma.

In addition, when there is a factor that increases or decreases amount of mRNA in milk, and increase or decrease of the amount of the mRNA is suppressed by ingestion of a certain diet, the diet has an action of decreasing or increasing the amount of the mRNA.

The diet may consist of a single substance or may be a composition, so long as it can be orally ingested. Further, “before ingestion” and “after ingestion” may mean “before and after one time of ingestion of diet”, or “before and after two or more times of ingestion of diet”. Further, two or more times of ingestion of diet may be two or more times of ingestion of the same diet, or ingestion of two or more kinds of diets.

The diet may be ingested according to a planned scheme or freely ingested. In the latter case, correlation of the diet and mRNA profile in milk can be examined by hearing content of ingested diet in the case of human. When the diet is ingested or administered according to a planned scheme, the diet can be considered as a “test sample”. The diet may be a usual diet or a usual diet containing a test substance. Amount of diet to be ingested, time of ingestion, number of times of ingestion and so forth are not particularly limited.

If a diet that increases amount of mRNA in milk is chosen, a substance that is contained in the diet and increases amount of the mRNA in milk can be identified in the same manner as that mentioned above. Further, if a diet that decreases amount of mRNA in milk is chosen, a substance that is contained in the diet and decreases amount of the mRNA in milk can be identified in the same manner as that mentioned above.

If a diet or a substance that increases or decreases amount of mRNA in milk is identified, a diet that increases or decreases amount of the mRNA in milk can be designed.

That is, a diet or a substance contained in the diet of which ingestion increases amount of mRNA of the same kind as that of mRNA of which amount in milk of a rat increases when a Bifidobacterium is orally administered to the rat, among mRNAs in milk, is preferred for production of milk having an immunostimulating action. Further, a diet or a substance contained in the diet of which ingestion decreases amount of mRNA of the same kind as that of mRNA of which amount in milk of a rat decreases when a Bifidobacterium is orally administered to the rat, among mRNAs in milk, is also preferred for production of milk having an immunostimulating action. Further, it is considered that these diets or substances are unsuitable for production of milk having an immunosuppressive action.

On the other hand, a diet or a substance contained in the diet of which ingestion decreases amount of mRNA of the same kind as that of mRNA of which amount in milk of a rat increases when a Bifidobacterium is orally administered to the rat, among mRNAs in milk, is preferred for production of milk having an immunosuppressive action. Further, a diet or a substance contained in the diet of which ingestion increases amount of mRNA of the same kind as that of mRNA of which amount in milk of a rat decreases when a Bifidobacterium is orally administered to the rat, among mRNAs in milk, is also preferred for production of milk having an immunosuppressive action. Further, it is considered that these diets or substances are unsuitable for production of milk having an immunostimulating action.

Screening for a diet or a substance providing production of breast milk having an immunoregulatory action, or a diet or a substance unsuitable for production of breast milk having an immunoregulatory action can be performed as described above. As shown in the examples described later, when a Bifidobacterium (Bifidobacterium longum) was orally administered to rats, amounts of 562 kinds of mRNAs increased, and amounts of 325 kinds of mRNAs decreased.

It is known that Bifidobacteria function as probiotics, and have, in particular, an immunoregulatory action. Therefore, the fact that the administration of the Bifidobacterium increased amounts of mRNAs in milk also supports the involvement of mRNAs in milk in immunoregulation. Demonstration of change in amounts of mRNAs in milk induced by administration of the Bifidobacterium, i.e., correlation of the Bifidobacterium and mRNA profile, shows that the screening method of the present invention is feasible. Further, although there were also mRNAs of which amounts in milk were not changed by administration of the Bifidobacterium, a possibility that amounts of those mRNAs may be increased or decreased by another kind of diet or a substance contained therein is not denied.

As probiotic functions of Bifidobacteria, there are known prophylaxis or amelioration of respiratory tract infection, acute infectious diarrhea, antibiotic-associated diarrhea, Clostridium dificile-associated diarrhea, necrotizing enterocolitis, traveler's diarrhea, Helicobacter pylori infection, and so forth (The Journal of Nutrition, 2010 March; 140(3):698S-712S. Epub 2010 Jan. 27). It is suggested that mRNA of which amount in milk is increased by administration of Bifidobacteria not only regulates immunity, but also exhibits functions similar to the aforementioned probiotic functions in mammals that ingested them.

By giving a diet or a substance that increases amount of mRNA in milk functioning in an immunostimulating manner chosen as described above to a mammal, and collecting milk from the mammal, milk having an immunostimulating action or milk of which immunostimulating action is enhanced can be obtained. Further, by reducing or avoiding ingestion by a mammal of a diet or a substance that decreases amount of mRNA in milk functioning in an immunostimulating manner chosen as described above, an immunostimulating action of milk can be enhanced, or decrease of an immunostimulating action can be prevented. Ingestion of a diet or a substance that increases or decreases amount of mRNA in milk, and reduction or avoidance of ingestion of a diet or a substance that decreases or increases amount of mRNA in milk may be used in combination.

Further, by giving a diet or a substance that increases amount of mRNA in milk functioning in an immunosuppressive manner chosen as described above to a mammal, and collecting milk from the mammal, milk having an immunosuppressive action or milk of which immunostimulating action is decreased can be obtained. Further, by reducing or avoiding ingestion by a mammal of a diet or a substance that decreases amount of mRNA in milk functioning in an immunosuppressive manner chosen as described above, an immunosuppressive action of milk can be enhanced, or decrease of an immunosuppressive action can be prevented. Ingestion of a diet or a substance that decreases or increases amount of mRNA in milk, and reduction or avoidance of ingestion of a diet or a substance that increases or decreases amount of mRNA in milk may be used in combination.

By processing milk having an immunoregulatory action obtained as described above, a dairy product having an immunoregulatory action can be produced.

Type of the dairy product is not particularly limited, so long as mRNA can exist in it with maintaining the functions thereof, and examples include processed milk, infant formula, milk beverages, powdered infant formula, fermented milk, cream, butter, cheese, ice cream, and so forth. As the dairy product, a dairy product for infants or little children is preferred.

According to the present invention, there was demonstrated presence in milk of mRNAs, especially mRNAs which have been known to participate in enhancement of immunity, such as development of immunity, antiallergy, anti-inflammation, and defense against infection. In addition, it is well known that breast milk gives an immunostimulating action to an infant who ingested it. Therefore, it is rationally predicted that mRNA participating in immunoregulation can regulate immunity of organism such as human who ingested it. Therefore, it is considered that transfer of various mRNAs from a mother to an infant is extremely significant for, in particular, infants having an underdeveloped immune system.

Another aspect of the present invention is a method for producing a composition for oral ingestion having an immunostimulating action, which comprises adding a mRNA of which amount in milk of a rat increases when a Bifidobacterium is orally administered to the rat to a base for a composition for oral ingestion. Examples of the mRNA include those mentioned in Tables 1 to 14 mentioned later.

Still another aspect of the present invention is a method for producing a composition for oral ingestion having an immunosuppressive action, which comprises adding a mRNA of which amount in milk of a rat decreases when a Bifidobacterium is orally administered to the rat to a base for a composition for oral ingestion. Examples of the mRNA include those mentioned in Tables 15 to 22 mentioned later.

The mRNA may consist of a single kind of mRNA or arbitrary two or more kinds of mRNAs.

The base for composition for oral ingestion is not particularly limited so long as an orally ingestible or administrable base in which mRNA can exist with maintaining functions thereof is chosen, and examples include foods, drinks, drug bases, feeds, and so forth.

Foods may be in any form, and include drinks. Foods include foods for adults, foods for infants, foods for little children, and so forth.

Examples of the foods for adults include enteral nutrients, fluid diets such as concentrated fluid diets, nutritional supplementary foods, and so forth.

Examples of the foods for infants or the foods for little children include, for example, modified milks (for example, infant formula, infant formula for low birth weight infants, follow-on formula, etc. as well as infant formula for allergic infants, non-lactose milk, special milk for inborn errors of metabolism infants, etc., and dried milk prepared from these), powders for supplement of breast milk or powdered infant formula, baby food, and so forth.

The infant formula referred to here are foods produced by using milk or dairy products as main raw materials, and adding nutrients required for infants, and are mainly used as alternative food for breast milk in infancy, and as alternative food for breast milk or nutritional complementary food in childhood. Other examples thereof include foods produced for the purpose of contributing to nutritional ingestion suitable for infants with a specific inherent or acquired disease.

mRNA is relatively resistant to freeze-thaw, low pH such as acidic condition of pH 2, and RNases (Hata, T. et al., Biochem. Biophys. Res. Commun., 396:528-533, 2010), and thus is suitable as an active ingredient to be added to foods. The stability at a low pH suggests that mRNA molecules are resistant to the infant's intragastric environment, and can be absorbed by the intestinal tract, which is one of the main immune organs of infants, and thus they can affect the immune system of infants. Further, storage and freeze-thaw of breast milk do not denature mRNA, and this is nutritionally important for low birth weight infants and hospitalized infants, who are usually given cryopreserved breast milk. Furthermore, the resistance of mRNA to RNases suggests that mRNA may exist in a complex such as exosome and microvesicle in breast milk. Similarly, when mRNA is added to a composition for oral ingestion, mRNA may be micellized by using, for example, lipid bilayer or the like.

Although content of mRNA in the composition for oral ingestion is not particularly limited, and may be appropriately chosen, it is, for example, 10 to 10,000 ng/ml, preferably 20 to 10,000 ng/ml, more preferably 50 to 10,000 ng/ml, in total. Further, amount of mRNA to be ingested is, for example, 5 μg to 120 mg/day, preferably 10 μg to 120 mg/day, more preferably 25 μg to 120 mg/day, in total.

mRNA can be prepared by, for example, gene engineering using a cell introduced with an expression vector that expresses a corresponding gene.

Further, mRNA can also be prepared by chemical synthesis. That is, mRNA can be obtained by synthesizing a sense strand and an antisense strand and annealing them.

Further, mRNA can also be prepared from breast milk or a fractionation product thereof containing mRNA. For example, mRNA that functions in an immunostimulating manner, for example, a mRNA of which amount in milk of a rat increases when a Bifidobacterium is orally administered to the rat, can be collected from breast milk of a mammal given a diet or a substance that increases amount of the mRNA in milk. Further, mRNA that functions in an immunosuppressive manner, for example, a mRNA of which amount in milk of a rat decreases when a Bifidobacterium is orally administered to the rat, can be collected from breast milk of a mammal given a diet or a substance that increases amount of the mRNA in milk. mRNA may be a purified product or a crude product. mRNA that functions in an immunostimulating manner may contain a small amount of mRNA that functions in an immunosuppressive manner, so long as the immunostimulating action of the mRNA is not degraded. Further, mRNA that functions in an immunosuppressive manner may contain a small amount of mRNA that functions in an immunostimulating manner, so long as the immunosuppressive action of the mRNA is not degraded.

When the composition for oral ingestion of the present invention is a pharmaceutical agent, the composition can be prepared by combining mRNA with pharmaceutically acceptable carriers for oral administration. The form of the pharmaceutical preparation is not particularly limited, and examples include tablet, pill, powder, solution, suspension, emulsion, granule, capsule, syrup, and so forth. For the formulation, additives widely used for usual pharmaceutical agents as pharmaceutical carriers for oral administration such as excipients, binders, disintegrating agents, lubricants, stabilizers, corrigents, diluents, and surfactants can be used. Further, unless the effect of the present invention is degraded, mRNA may be used together with another drug having an immunoregulatory action.

Although amount of mRNA contained in the pharmaceutical agent is not particularly limited, and can be appropriately chosen, it is, for example, 2 μg/kg to 2 mg/kg, preferably 4 μg/kg to 2 mg/kg, more preferably 10 μg/kg to 2 mg/kg, in total.

When the composition for oral ingestion is a food, it may be for any of various uses utilizing an immunostimulating action. Examples of the use include, for example, uses as foods suitable for persons showing reduced resistance, uses as foods or drinks useful for reduction and elimination of risk factors of various diseases caused by immune depression, and so forth.

The foods or drinks of the present invention can be marketed as foods attached with an indication describing that the foods or drinks are used for immunoregulation.

The aforementioned term “indication” includes all actions for informing consumers the aforementioned use, and any indications reminding or analogizing the aforementioned use fall within the scope of the “indication” of the present invention regardless of purpose, content, objective article, medium etc. of the indication. However, the indication is preferably made with an expression that allows consumers to directly recognize the aforementioned use. Specific examples include actions of indicating the aforementioned use on goods or packages of goods relating to the food of the present invention, actions of assigning, delivering, displaying for the purpose of assigning or delivering or importing such goods or packages of goods on which the aforementioned use is indicated, displaying or distributing advertisements, price lists or business papers relating the goods on which the aforementioned use is indicated, or providing information including those as contents with indicating the aforementioned use by an electromagnetic method (Internet etc.) and so forth.

The indication is preferably an indication approved by the administration etc. (for example, an indication in a form based on an approval, which is qualified on the basis of any of various legal systems provided by the administration), and it is particularly preferably an indication on advertisement materials at the sales spots such as packages, containers, catalogs, pamphlets and POPs (point of purchase advertising), other documents and so forth.

Examples of the indication further include, for example, indications as health food, functional food, enteric nutritive food, food for special dietary uses, food with nutrient function claims, quasi-drug and so forth as well as indications approved by the Ministry of Health, Labor and Welfare, for example, indications approved on the basis of the system of food for specified health uses and similar systems. Examples of the latter include indications as food for specified health uses, indications as food for specified health uses with qualified health claims, indications of influence on body structures and functions, indications of reduction of disease risk claims and so forth, and more precisely, typical examples include indications as food for specified health uses (especially indications of use for health) provided in the enforcement regulations of Health Promotion Law (Japanese Ministry of Health, Labor and Welfare, Ministerial ordinance No. 86, Apr. 30, 2003) and similar indications.

As shown in the examples, rat milk contained many kinds of mRNAs derived from genes coding for hormones such as growth hormone, cholecystokinin, and gastrin, growth factors such as epidermal growth factor, fibroblast growth factor, platelet-derived growth factor, and vascular endothelial growth factor, enzymes such as fatty acid synthase, lipoprotein lipase, and acyl-CoA synthase, and so forth. It is considered that these proteins have an action of promoting growth of children (baby mammals). Therefore, the present invention can be similarly applied to screening for a diet or a substance that provides production of breast milk having an enhanced action of promoting growth of children (baby mammals), a method for producing milk or a dairy product having such an action, and a method for producing a composition for oral ingestion having such an action.

### EXAMPLES

Hereafter, the present invention will be further specifically explained with reference to examples. However, the present invention is not limited to the following examples.

### Example 1

### Analysis of mRNA in Rat Milk

Three F344 rats on day 14 of gestation were purchased. All the purchased rats gave birth on day 21 of gestation, and they were milked 2, 9 and 16 days after the delivery under ether anesthesia to collect milk. Further, they were dissected 21 days after the delivery, which corresponded to the weaning day, and blood was collected from the inferior vena cava.

Each milk sample was centrifuged at 1,200×g and 4° C. for 10 minutes, and the lipid layer and cell debris were removed.

Then, each sample was centrifuged twice at 21,500×g and 4° C. for 30 minutes, further centrifuged once under the same conditions for 1 hour to remove the casein fraction, and passed through membranes of 0.65 μm, 0.45 μm and 0.22 μm to remove cell debris and insoluble matter and thereby obtain a milk serum sample.

The blood obtained by the blood collection was centrifuged at 1,200×g and 4° C. for 10 minutes, and passed through membranes of 0.65 μm, 0.45 μm and 0.22 μm to remove cell debris and insoluble matter and thereby obtain a blood serum sample.

Total RNAs were obtained from the obtained milk serum sample and the blood serum sample by using miRNeasy Mini Kit (Qiagen). Since the blood serum sample contained only a small amount of RNA, the blood sera of three rats were combined, and the total RNA was obtained from the combined blood serum.

After quality of each obtained RNA solution was confirmed by using BioAnalyzer, 100 ng of RNA was used to synthesize cRNA by using Low RNA Input Liner Amplification Kit (Agilent Technologies). At the time of this synthesis, CPT labeled with a cyanine dye (Cyanine 3-CPT) was incorporated into cRNA to attain fluorescence labeling of cRNA. The labeled cRNA was added to a hybridization buffer, allowed to hybridize on Whole Rat Genome Oligo Microarray (4×44 K, Agilent Technologies) for 17 hours, washed, and then imaged with Agilent Microarray Scanner (Agilent Technologies), and the fluorescent signals of the spots were numerically represented by using Feature Extraction Software. The results were analyzed by using GeneSpring GX11.0 (Agilent Technologies).

As a result, in the milk serum sample, mRNAs corresponding to 10,948 kinds of probes were detected, and in the blood serum sample, mRNAs corresponding to 2,361 kinds of probes were detected. The mRNAs detected in the milk serum sample included many mRNAs derived from genes coding for proteins relating to the immune system, such as cytokines, chemokines, and antimicrobial peptides. In fact, when biological interpretation was performed by using Ingenuity Pathway Analysis (Ingenuity Systems), it was demonstrated that many mRNAs relating to pathways of “infection mechanism” or “infectious disease” were included. An action of defending children (baby mammals) from infection is known for milk, and interestingly, the defense from infection has also been suggested on the mRNA level. In addition, many mRNAs derived from genes coding for proteins that promote growth of children (baby mammals) such as hormones, growth factors, and enzymes were also contained.

### Example 2

### Identification of Diet or Substance Providing Production of Milk Having Immunoregulatory Action

SD rats on day 9 to 10 of gestation were purchased, and during the period from day 13 to day 20 of gestation, a suspension in which cells of a Bifidobacterium, Bifidobacterium longum BB536 (ATCC BAA-999), were suspended in PBS (phosphate buffered saline) (1×10⁹ cfu/ml) was orally administered everyday to the rats of test group (n=3) in a volume of 1 ml/day per rat.

Further, 1 ml/day per rat of PBS was administered everyday to the rats of control group (n=3). The B. longum ATCC BAA-999 strain can be purchased from American Type Culture Collection (Address: 12301 Parklawn Drive, Rockville, Md. 20852, United States of America).

All the rats gave birth on day 21 of gestation, and they were milked under ether anesthesia one day after the birth. Each obtained milk sample was centrifuged at 1,200×g and 4° C. for 10 minutes, and the lipid layer and cell debris were removed.

Then, each sample was centrifuged twice at 21,500×g and 4° C. for 30 minutes, further centrifuged once under the same conditions for 1 hour to remove the casein fraction, and passed through membranes of 0.65 μm, 0.45 μm and 0.22 μm to remove cell debris and insoluble matter and thereby obtain a milk serum sample.

Total RNA was obtained from the obtained milk serum sample by using miRNeasy Mini Kit (Qiagen). By using 100 ng of the obtained RNA sample, detection of mRNAs and analysis of expression amounts thereof were conducted in the same manner as that of Example 1.

As a result, number of probes corresponding to mRNAs confirmed in the test group and the control group was 10,239.

Further, the expression amounts in the Bifidobacterium longum BB536 strain-administered group and the control group were compared, and evaluated by the Mann-Whitney U-test. As a result, at a probability level of P<0.05, number of mRNAs that increased 1.5 times or more was 562, and number of mRNAs that decreased to 0.67 times or less was 325. These mRNAs and rate of change (RC) in expression amounts thereof are shown in Tables 1 to 22. The mRNAs of which expression amounts increased are mentioned in Tables 1 to 14, and the mRNAs of which expression amounts decreased are mentioned in Tables 15 to 22.

TABLE 1

| ProbeName | RC | GeneSymbol | EntrezGene ID | GeneName |
|---|---|---|---|---|
| A_44_P235892 | 7.30 | Cyp24a1 | 25279 | cytochrome P450, family 24, subfamily a, polypeptide 1 |
| A_44_P468182 | 6.80 | Dnm1 | 140694 | dynamin 1 |
| A_44_P311647 | 6.64 | Dpysl2 | 25416 | dihydropyrimidinase-like 2 |
| A_42_P619403 | 6.47 | Fam89a | 361441 | family with sequence similarity 89, member A |
| A_44_P1041539 | 6.05 | Arhgap22 | 306279 | Rho GTPase activating protein 22 |
| A_43_P17699 | 5.86 | Fbxl5 | 305424 | F-box and leucine-rich repeat protein 5 |
| A_44_P220008 | 5.85 | LOC304396 | 304396 | similar to hypothetical protein DKFZp434K1815 |
| A_44_P398142 | 5.40 | Btg2 | 29619 | BTG family, member 2 |
| A_44_P253782 | 5.29 | Csnk1g1 | 64086 | casein kinase 1, gamma 1 |
| A_44_P607813 | 5.19 | Mll1 | 315606 | myeloid/lymphoid or mixed-lineage leukemia 1 |
| A_44_P496025 | 4.78 | Eml4 | 313861 | echinoderm microtubule associated protein like 4 |
| A_44_P494147 | 4.76 | Nelf | 117536 | nasal embryonic LHRH factor |
| A_43_P12247 | 4.74 | Aldh1l1 | 64392 | aldehyde dehydrogenase 1 family, member L1 |
| A_44_P392004 | 4.68 | Boc | 360715 | biregional cell adhesion molecule-related/down-regulated by |
| | | | | oncogenes (Cdon) binding protein |
| A_44_P347420 | 4.67 | Usp43_predicted | 100147704 | ubiquitin specific protease 43 |
| A_44_P442802 | 4.60 | Rassf5 | 54355 | Ras association (RalGDS/AF-6) domain family member 5 |
| A_44_P1038694 | 4.59 | Man2a1 | 25478 | mannosidase, alpha, class 2A, member 1 |
| A_42_P559737 | 4.42 | Srgn | 56782 | serglycin |
| A_43_P10254 | 4.41 | Gbf1 | 309451 | golgi-specific brefeldin A resistant guanine nucleotide |
| | | | | exchange factor 1 |
| A_44_P869362 | 4.38 | RGD1562551 | 311760 | similar to C20orf82 |
| A_43_P18573 | 4.37 | Lrba | 361975 | LPS-responsive vesicle trafficking, beach and anchor |
| | | | | containing |
| A_44_P520159 | 4.34 | P2ry2 | 29597 | purinergic receptor P2Y, G-protein coupled, 2 |
| A_42_P623909 | 4.21 | Bcl2l1 | 24888 | Bcl2-like 1 |
| A_44_P251673 | 4.18 | Rbm14 | 170900 | RNA binding motif protein 14 |
| A_44_P242339 | 4.13 | RGD1307365 | 300880 | similar to KIAA1009 protein |
| A_43_P21138 | 4.11 | Tecpr1 | 304285 | tectonin beta-propeller repeat containing 1 |
| A_43_P10399 | 4.11 | Kctd9 | 364410 | potassium channel tetramerisation domain containing 9 |
| A_44_P545478 | 4.10 | Pkig | 266709 | protein kinase inhibitor, gamma |
| A_43_P19970 | 4.09 | RGD1308139 | 360563 | similar to RIKEN cDNA 1200014J11 |
| A_43_P22368 | 4.05 | Klhl35 | 308850 | kelch-like 35 (Drosophila) |
| A_44_P176342 | 4.03 | Gab1 | 361388 | GRB2-associated binding protein 1 |
| A_44_P253196 | 4.02 | Arid5b | 309728 | AT rich interactive domain 5B (Mrf1 like) |
| A_44_P871726 | 4.01 | Zfp157 | 360775 | zinc finger protein 157 |
| A_43_P10581 | 4.00 | Mafk | 246760 | v-maf musculoaponeurotic fibrosarcoma oncogene homolog K |
| | | | | (avian) |
| A_42_P700288 | 3.93 | Trim47 | 690374 | tripartite motif-containing 47 |
| A_44_P471166 | 3.89 | Ddx21 | 317399 | DEAD (Asp-Glu-Ala-Asp) box polypeptide 21 |
| A_44_P471018 | 3.86 | Tiparp | 310467 | TCDD-inducible poly(ADP-ribose) polymerase |
| A_44_P1042876 | 3.86 | Hspa1b | 294254 | heat shock 70 kD protein 1B (mapped) |
| A_44_P977318 | 3.80 | Tcfcp2l1 | 304741 | transcription factor CP2-like 1 |
| A_43_P15281 | 3.78 | Nr3c2 | 25672 | nuclear receptor subfamily 3, group C, member 2 |
| A_43_P12908 | 3.76 | Klf4 | 114505 | Kruppel-like factor 4 (gut) |

TABLE 2

| ProbeName | RC | GeneSymbol | EntrezGene ID | GeneName |
|---|---|---|---|---|
| A_44_P547957 | 3.76 | Dhx8 | 287727 | DEAH (Asp-Glu-Ala-His) box polypeptide 8 |
| A_44_P140092 | 3.75 | Myh14 | 308572 | myosin, heavy chain 14 |
| A_44_P317388 | 3.73 | Mcam | 78967 | melanoma cell adhesion molecule |
| A_44_P729514 | 3.71 | Dusp3 | 498003 | dual specificity phosphatase 3 |
| A_44_P674018 | 3.71 | Acer2 | 313339 | alkaline ceramidase 2 |
| A_44_P668591 | 3.70 | L3mbtl3 | 309550 | l(3)mbt-like 3 (Drosophila) |
| A_44_P468489 | 3.68 | Fcho2 | 309129 | FCH domain only 2 |
| A_42_P521707 | 3.66 | RT1-N3 | 24750 | RT1 class Ib, locus N3 |
| A_44_P879104 | 3.63 | Ash1l | 310638 | ash1 (absent, small, or homeotic)-like (Drosophila) |
| A_44_P294467 | 3.59 | Paxip1 | 311944 | PAX interacting (with transcription-activation domain) protein 1 |
| A_44_P170778 | 3.57 | Irgq | 292708 | immunity-related GTPase family, Q |
| A_44_P199211 | 3.55 | Plcb1 | 24654 | phospholipase C, beta 1 (phosphoinositide-specific) |
| A_42_P702550 | 3.54 | Sts | 24800 | steroid sulfatase |
| A_44_P166068 | 3.54 | Spetex-2D | 364260 | Spetex-2D protein |
| A_44_P389462 | 3.54 | Zmym3 | 317260 | zinc finger, MYM-type 3 |
| A_43_P11614 | 3.49 | Anxa1 | 25380 | annexin A1 |
| A_44_P384677 | 3.45 | Rtn4ip1 | 309912 | reticulon 4 interacting protein 1 |
| A_44_P254366 | 3.43 | Gngt1 | 680149 | guanine nucleotide binding protein (G protein), gamma |
| | | | | transducing activity polypeptide 1 |
| A_42_P615851 | 3.43 | Srcap | 361652 | Snf2-related CREBBP activator protein |
| A_44_P213208 | 3.41 | Otud4 | 307774 | OTU domain containing 4 |
| A_43_P17271 | 3.38 | RGD1307569 | 360695 | similar to Protein C21orf63 homolog precursor |
| A_43_P21650 | 3.36 | Adpgk | 315722 | ADP-dependent glucokinase |
| A_44_P135224 | 3.36 | Plk3 | 58936 | polo-like kinase 3 (Drosophila) |
| A_44_P1057137 | 3.34 | Phf1 | 294287 | PHD finger protein 1 |
| A_43_P12878 | 3.33 | St14 | 114093 | suppression of tumorigenicity 14 (colon carcinoma) |
| A_44_P1044517 | 3.32 | N4bp1 | 291921 | Nedd4 binding protein 1 |
| A_43_P17298 | 3.30 | Naaa | 497009 | N-acylethanolamine acid amidase |
| A_43_P13203 | 3.29 | Expi | 171059 | extracellular proteinase inhibitor |
| A_44_P392234 | 3.28 | Fgf7 | 29348 | fibroblast growth factor 7 |
| A_44_P651749 | 3.26 | RGD1562492 | 499151 | similar to Orphan sodium- and chloride-dependent |
| | | | | neurotransmitter transporter NTT5 (Solute carrier family 6 |
| | | | | member 16) |
| A_43_P21455 | 3.26 | Lama2 | 309368 | laminin, alpha 2 |
| A_44_P342075 | 3.25 | Ahnak | 191572 | AHNAK nucleoprotein |
| A_44_P414757 | 3.25 | Sec3l1 | 305287 | SEC3-like 1 (S. cerevisiae) |
| A_44_P635769 | 3.25 | Mks1 | 287612 | Meckel syndrome, type 1 |
| A_43_P16750 | 3.24 | Prodh | 680409 | proline dehydrogenase |
| A_42_P831092 | 3.23 | Baz1b | 368002 | bromodomain adjacent to zinc finger domain, 1B |
| A_42_P658005 | 3.21 | R3hdm1 | 304763 | R3H domain containing 1 |
| A_44_P869639 | 3.20 | Setd5 | 297514 | SET domain containing 5 |
| A_44_P1050325 | 3.20 | Rhebl1 | 359959 | Ras homolog enriched in brain like 1 |
| A_44_P175461 | 3.19 | Kdm5b | 304809 | lysine (K)-specific demethylase 5B |
| A_43_P18798 | 3.19 | Fbxw8 | 304522 | F-box and WD repeat domain containing 8 |
| A_42_P625263 | 3.19 | Epb4.1l4a | 307514 | erythrocyte protein band 4.1-like 4a |
| A_44_P690201 | 3.15 | Lpin2 | 316737 | lipin 2 |
| A_44_P515197 | 3.14 | Cxcl2 | 114105 | chemokine (C-X-C motif) ligand 2 |

TABLE 3

| ProbeName | RC | GeneSymbol | EntrezGene ID | GeneName |
|---|---|---|---|---|
| A_44_P522388 | 3.14 | Nr2c2 | 50659 | nuclear receptor subfamily 2, group C, member 2 |
| A_44_P408050 | 3.11 | Ckap4 | 362859 | cytoskeleton-associated protein 4 |
| A_44_P284753 | 3.10 | Ankrd1 | 27064 | ankyrin repeat domain 1 (cardiac muscle) |
| A_44_P172604 | 3.09 | Ugcg | 83626 | UDP-glucose ceramide glucosyltransferase |
| A_42_P735417 | 3.08 | Fcgr2b | 289211 | Fc fragment of IgG, low affinity IIb, receptor (CD32) |
| A_44_P668137 | 3.07 | Sf3a1 | 305479 | splicing factor 3a, subunit 1 |
| A_44_P503570 | 3.07 | Parg | 83507 | poly (ADP-ribose) glycohydrolase |
| A_44_P905278 | 3.06 | Heatr6 | 497972 | HEAT repeat containing 6 |
| A_42_P671285 | 3.06 | Ctdspl | 301056 | CTD (carboxy-terminal domain, RNA polymerase II, |
| | | | | polypeptide A) small phosphatase-like |
| A_44_P276782 | 3.04 | Tp53bp1 | 296099 | tumor protein p53 binding protein 1 |
| A_42_P772136 | 3.02 | Fam60a | 686611 | family with sequence similarity 60, member A |
| A_43_P12150 | 3.02 | Pik3r2 | 29741 | phosphoinositide-3-kinase, regulatory subunit 2 (beta) |
| A_44_P157078 | 3.00 | RGD1305089 | 299050 | similar to 1110008L16Rik protein |
| A_44_P930917 | 3.00 | Arhgef5 | 140898 | Rho guanine nucleotide exchange factor (GEF) 5 |
| A_44_P281155 | 2.98 | Nek7 | 360850 | NIMA (never in mitosis gene a)-related kinase 7 |
| A_44_P926632 | 2.95 | Rp2 | 367714 | retinitis pigmentosa 2 homolog (human) |
| A_44_P477135 | 2.94 | Vhl | 24874 | von Hippel-Lindau tumor suppressor |
| A_43_P17943 | 2.94 | Usp31 | 308959 | ubiquitin specific peptidase 31 |
| A_44_P1011373 | 2.93 | Camsap1 | 296580 | calmodulin regulated spectrin-associated protein 1 |
| A_44_P914747 | 2.93 | RGD1308759 | 290668 | similar to KIAA0892 protein |
| A_44_P854689 | 2.90 | Ldlrap1 | 500564 | low density lipoprotein receptor adaptor protein 1 |
| A_44_P437641 | 2.89 | RGD1359108 | 313155 | similar to RIKEN cDNA 3110043O21 |
| A_44_P681569 | 2.88 | Sp1 | 24790 | Sp1 transcription factor |
| A_44_P623197 | 2.88 | Mast4 | 310040 | microtubule associated serine/threonine kinase family member 4 |
| A_44_P184726 | 2.88 | Rela | 309165 | v-rel reticuloendotheliosis viral oncogene homolog A (avian) |
| A_44_P791631 | 2.88 | Nhlrc3 | 310416 | NHL repeat containing 3 |
| A_43_P22909 | 2.87 | Intu | 361938 | inturned planar cell polarity effector homolog (Drosophila) |
| A_44_P138800 | 2.86 | Gcap14 | 306306 | granule cell antiserum positive 14 |
| A_44_P185015 | 2.84 | Spetex-2B | 497084 | Spetex-2B protein |
| A_44_P959960 | 2.84 | Cdc42se2 | 691031 | CDC42 small effector 2 |
| A_44_P485388 | 2.83 | Chd7 | 312974 | chromodomain helicase DNA binding protein 7 |
| A_44_P306964 | 2.83 | Rbms1 | 362138 | RNA binding motif, single stranded interacting protein 1 |
| A_44_P624151 | 2.82 | Invs | 313228 | inversin |
| A_44_P321488 | 2.82 | LOC100294508 | 100294508 | dyslexia susceptibility 2-like |
| A_44_P989302 | 2.81 | Kif2a | 84391 | kinesin family member 2A |
| A_44_P401515 | 2.81 | Exoc6 | 50556 | exocyst complex component 6 |
| A_43_P18884 | 2.81 | Zbtb1 | 314246 | zinc finger and BTB domain containing 1 |
| A_44_P414460 | 2.80 | Rnd3 | 295588 | Rho family GTPase 3 |
| A_44_P122401 | 2.79 | Rb1cc1 | 312927 | RB1-inducible coiled-coil 1 |
| A_44_P381917 | 2.78 | Sphk1 | 170897 | sphingosine kinase 1 |

TABLE 4

| ProbeName | RC | GeneSymbol | EntrezGene ID | GeneName |
|---|---|---|---|---|
| A_44_P402641 | 2.78 | Ugt1a2 | 396527 | UDP glucuronosyltransferase 1 family, polypeptide A2 |
| A_44_P401131 | 2.76 | Noxo1 | 302976 | NADPH oxidase organizer 1 |
| A_44_P173657 | 2.76 | Mllt6 | 303504 | myeloid/lymphoid or mixed-lineage leukemia (trithorax |
| | | | | homolog, Drosophila); translocated to, 6 |
| A_44_P102001 | 2.76 | Thumpd2 | 313851 | THUMP domain containing 2 |
| A_44_P194172 | 2.74 | Ptpn4 | 246116 | protein tyrosine phosphatase, non-receptor type 4 |
| A_44_P1060513 | 2.74 | Thex1 | 361159 | three prime histone mRNA exoribonuclease 1 |
| A_44_P874704 | 2.74 | S100a8 | 116547 | S100 calcium binding protein A8 |
| A_44_P363229 | 2.71 | Grk5 | 59075 | G protein-coupled receptor kinase 5 |
| A_44_P825195 | 2.71 | Trove2 | 304833 | TROVE domain family, member 2 |
| A_44_P684740 | 2.70 | Pcdh17 | 306055 | protocadherin 17 |
| A_44_P445972 | 2.70 | Med13 | 303403 | mediator complex subunit 13 |
| A_44_P522047 | 2.70 | LOC684871 | 684871 | similar to Protein C8orf4 (Thyroid cancer protein 1) (TC-1) |
| A_44_P295467 | 2.69 | RGD1305350 | 313699 | similar to RIKEN cDNA 2510039O18 |
| A_44_P365882 | 2.69 | Fam82a2 | 311328 | family with sequence similarity 82, member A2 |
| A_44_P161602 | 2.68 | Rab43 | 500249 | RAB43, member RAS oncogene family |
| A_44_P1031034 | 2.67 | Clip1 | 65201 | CAP-GLY domain containing linker protein 1 |
| A_44_P976816 | 2.66 | Ankrd57 | 503306 | ankyrin repeat domain 57 |
| A_44_P344928 | 2.65 | RGD1563375 | 366392 | similar to small nuclear ribonucleoparticle-associated protein |
| A_42_P538084 | 2.64 | Crim1 | 298744 | cysteine rich transmembrane BMP regulator 1 (chordin like) |
| A_43_P15220 | 2.64 | Celsr2 | 83465 | cadherin, EGF LAG seven-pass G-type receptor 2 (flamingo |
| | | | | homolog, Drosophila) |
| A_43_P12569 | 2.63 | Idh1 | 24479 | isocitrate dehydrogenase 1 (NADP+), soluble |
| A_44_P372148 | 2.63 | Lrrc61 | 500111 | leucine rich repeat containing 61 |
| A_44_P1034030 | 2.63 | Aff4 | 303132 | AF4/FMR2 family, member 4 |
| A_44_P520557 | 2.63 | Arhgap1 | 311193 | Rho GTPase activating protein 1 |
| A_44_P288187 | 2.63 | Itpr3 | 25679 | inositol 1,4,5-triphosphate receptor, type 3 |
| A_44_P440178 | 2.62 | Lhfp | 499615 | lipoma HMGIC fusion partner |
| A_44_P1005649 | 2.62 | Dnajc12 | 619393 | DnaJ (Hsp40) homolog, subfamily C, member 12 |
| A_44_P539680 | 2.62 | Garnl1 | 56785 | GTPase activating Rap/RanGAP domain-like 1 |
| A_42_P521902 | 2.61 | Gtf3c5 | 362095 | general transcription factor IIIC, polypeptide 5 |
| A_44_P134143 | 2.60 | Hspb8 | 113906 | heat shock protein B8 |
| A_43_P17471 | 2.59 | Chac1 | 362196 | ChaC, cation transport regulator homolog 1 (E. coli) |
| A_44_P208688 | 2.58 | Pof1b | 302328 | premature ovarian failure 1B |
| A_44_P370033 | 2.57 | Ptpre | 114767 | protein tyrosine phosphatase, receptor type, E |
| A_44_P667206 | 2.57 | Arl6ip1 | 293551 | ADP-ribosylation factor-like 6 interacting protein 1 |
| A_44_P473754 | 2.56 | Son | 304092 | Son DNA binding protein |
| A_44_P116300 | 2.56 | Ppargc1b | 291567 | peroxisome proliferator-activated receptor gamma, |
| | | | | coactivator 1 beta |
| A_44_P838798 | 2.55 | Med1 | 497991 | mediator complex subunit 1 |
| A_42_P660129 | 2.55 | Casp12 | 156117 | caspase 12 |
| A_44_P684579 | 2.52 | Zbtb39 | 299510 | zinc finger and BTB domain containing 39 |
| A_44_P187830 | 2.51 | Urb1 | 304104 | URB1 ribosome biogenesis 1 homolog (S. cerevisiae) |
| A_44_P915229 | 2.50 | Wdr89 | 314243 | WD repeat domain 89 |

TABLE 5

| ProbeName | RC | GeneSymbol | EntrezGene ID | GeneName |
|---|---|---|---|---|
| A_44_P336772 | 2.50 | Josd1 | 315134 | Josephin domain containing 1 |
| A_44_P358216 | 2.50 | Pank4 | 171053 | pantothenate kinase 4 |
| A_42_P597258 | 2.49 | Ska2 | 287598 | spindle and kinetochore associated complex subunit 2 |
| A_44_P148161 | 2.49 | Rrm1 | 685579 | ribonucleotide reductase M1 |
| A_43_P23126 | 2.48 | Klf11 | 313994 | Kruppel-like factor 11 |
| A_44_P426823 | 2.45 | Phc1 | 312690 | polyhomeotic-like 1 (Drosophila) |
| A_44_P774203 | 2.45 | Birc6 | 313876 | baculoviral IAP repeat-containing 6 |
| A_44_P283399 | 2.44 | Cask | 29647 | calcium/calmodulin-dependent serine protein kinase (MAGUK |
| | | | | family) |
| A_43_P21324 | 2.44 | Mesdc2 | 308796 | mesoderm development candidate 2 |
| A_44_P456968 | 2.43 | St5 | 308944 | suppression of tumorigenicity 5 |
| A_43_P21033 | 2.43 | Smyd5 | 312503 | SET and MYND domain containing 5 |
| A_44_P256534 | 2.43 | Heca | 308624 | headcase homolog (Drosophila) |
| A_44_P148370 | 2.42 | Plagl2 | 296281 | pleiomorphic adenoma gene-like 2 |
| A_42_P759427 | 2.42 | Strn4 | 308392 | striatin, calmodulin binding protein 4 |
| A_44_P581181 | 2.42 | Ftsjd1 | 292016 | FtsJ methyltransferase domain containing 1 |
| A_44_P729621 | 2.42 | Maml1 | 303101 | mastermind like 1 (Drosophila) |
| A_42_P684264 | 2.41 | RGD1561817 | 294917 | similar to Traf2 and NCK interacting kinase, splice variant 4 |
| A_44_P525229 | 2.40 | Anxa8 | 306283 | annexin A8 |
| A_44_P107423 | 2.40 | Nup153 | 25281 | nucleoporin 153 |
| A_42_P532103 | 2.40 | Anxa2 | 56611 | annexin A2 |
| A_44_P182061 | 2.40 | Trappc2 | 501550 | trafficking protein particle complex 2 |
| A_44_P653272 | 2.40 | Gpr155 | 311730 | G protein-coupled receptor 155 |
| A_42_P782490 | 2.39 | Zfhx3 | 307829 | zinc finger homeobox 3 |
| A_44_P729898 | 2.38 | Morc2 | 289736 | MORC family CW-type zinc finger 2 |
| A_44_P899288 | 2.38 | Lrsam1 | 311866 | leucine rich repeat and sterile alpha motif containing 1 |
| A_44_P210198 | 2.38 | Polg | 85472 | polymerase (DNA directed), gamma |
| A_44_P243534 | 2.35 | RGD1311307 | 361238 | similar to 1300014I06Rik protein |
| A_44_P182479 | 2.35 | Kcnk4 | 116489 | potassium channel, subfamily K, member 4 |
| A_44_P397423 | 2.35 | Tmcc3 | 314751 | transmembrane and coiled-coil domain family 3 |
| A_44_P381270 | 2.34 | RGD1562136 | 501195 | similar to D1Ertd622e protein |
| A_44_P258949 | 2.33 | Pde4b | 24626 | phosphodiesterase 4B, cAMP specific |
| A_44_P302613 | 2.33 | Lkap | 170946 | limkain b1 |
| A_44_P548507 | 2.33 | Chn1 | 84030 | chimerin (chimaerin) 1 |
| A_44_P853102 | 2.33 | Dusp16 | 297682 | dual specificity phosphatase 16 |
| A_43_P21031 | 2.32 | Pex5 | 312703 | peroxisomal biogenesis factor 5 |
| A_44_P191512 | 2.32 | Ns5atp9 | 300795 | NS5A (hepatitis C virus) transactivated protein 9 |
| A_44_P344181 | 2.32 | Map6 | 29457 | microtubule-associated protein 6 |
| A_44_P391057 | 2.32 | Angptl4 | 362850 | angiopoietin-like 4 |
| A_44_P287958 | 2.32 | Dnmt3b | 444985 | DNA (cytosine-5-)-methyltransferase 3 beta |
| A_44_P1032305 | 2.31 | Fert2 | 301737 | fer (fms/fps related) protein kinase, testis specific 2 |
| A_43_P19499 | 2.31 | Gzf1 | 311508 | GDNF-inducible zinc finger protein 1 |
| A_44_P340163 | 2.31 | Plcb4 | 25031 | phospholipase C, beta 4 |
| A_44_P395928 | 2.31 | Ap4m1 | 304344 | adaptor-related protein complex 4, mu 1 subunit |

TABLE 6

| ProbeName | RC | GeneSymbol | EntrezGene ID | GeneName |
|---|---|---|---|---|
| A_44_P787936 | 2.31 | Stat3 | 25125 | signal transducer and activator of transcription 3 |
| A_44_P1014018 | 2.31 | Rkhd1 | 299613 | ring finger (C3HC4 type) and KH domain containing 1 |
| A_44_P652103 | 2.30 | LOC691223 | 691223 | hypothetical protein LOC691223 |
| A_44_P457169 | 2.29 | Dcp2 | 291604 | DCP2 decapping enzyme homolog (S. cerevisiae) |
| A_44_P477322 | 2.29 | Rtn4 | 83765 | reticulon 4 |
| A_44_P1021361 | 2.29 | Tlr3 | 364594 | toll-like receptor 3 |
| A_44_P1022458 | 2.29 | Tubb6 | 307351 | tubulin, beta 6 |
| A_44_P298210 | 2.29 | Casp8 | 64044 | caspase 8 |
| A_44_P222799 | 2.28 | Nin | 299117 | ninein (GSK3B interacting protein) |
| A_44_P149087 | 2.28 | Fyn | 25150 | FYN oncogene related to SRC, FGR, YES |
| A_43_P17988 | 2.28 | Tmem77 | 362011 | transmembrane protein 77 |
| A_44_P396115 | 2.28 | Maml3 | 310405 | mastermind like 3 (Drosophila) |
| A_42_P589201 | 2.27 | Slc15a4 | 246280 | solute carrier family 15, member 4 |
| A_44_P170496 | 2.26 | Sqrdl | 691966 | sulfide quinone reductase-like (yeast) |
| A_44_P558636 | 2.26 | Ppp1r9b | 84686 | protein phosphatase 1, regulatory subunit 9B |
| A_44_P177448 | 2.26 | Pafah1b1 | 83572 | platelet-activating factor acetylhydrolase, isoform 1b, subunit 1 |
| A_44_P885147 | 2.26 | Def8 | 307973 | differentially expressed in FDCP 8 homolog (mouse) |
| A_44_P428997 | 2.25 | Fam164a | 310244 | family with sequence similarity 164, member A |
| A_44_P407894 | 2.25 | Znf746 | 312303 | zinc finger protein 746 |
| A_42_P473389 | 2.25 | Bmyc | 311807 | brain expressed myelocytomatosis oncogene |
| A_42_P680545 | 2.24 | RGD1305235 | 292267 | similar to RIKEN cDNA 1700052N19 |
| A_42_P489223 | 2.23 | Thada | 313865 | thyroid adenoma associated |
| A_44_P503022 | 2.23 | Hip1 | 192154 | huntingtin interacting protein 1 |
| A_44_P228839 | 2.23 | Pgrmc2 | 361940 | progesterone receptor membrane component 2 |
| A_44_P403621 | 2.22 | Sgk3 | 171498 | serum/glucocorticoid regulated kinase 3 |
| A_44_P117148 | 2.22 | Baz1a | 314126 | bromodomain adjacent to zinc finger domain, 1A |
| A_44_P607486 | 2.21 | Foxn3 | 314374 | forkhead box N3 |
| A_44_P257206 | 2.21 | Map3k12 | 25579 | mitogen activated protein kinase kinase kinase 12 |
| A_44_P618385 | 2.21 | Efhd2 | 298609 | EF-hand domain family, member D2 |
| A_44_P191055 | 2.21 | Pogz | 310658 | pogo transposable element with ZNF domain |
| A_44_P1030766 | 2.20 | Pcf11 | 361605 | PCF11, cleavage and polyadenylation factor subunit, homolog |
| | | | | (S. cerevisiae) |
| A_44_P112050 | 2.20 | RGD1560523 | 498211 | similar to S-adenosylmethionine synthetase gamma form |
| | | | | (Methionine adenosyltransferase) |
| A_44_P792035 | 2.19 | Mcart1 | 313241 | mitochondrial carrier triple repeat 1 |
| A_44_P382226 | 2.19 | Sox4 | 364712 | SRY (sex determining region Y)-box 4 |
| A_42_P484663 | 2.18 | Calcoco1 | 246047 | calcium binding and coiled coil domain 1 |
| A_43_P15113 | 2.18 | Myom1 | 316740 | myomesin 1 |
| A_42_P519018 | 2.17 | Mtmr9 | 282584 | myotubularin related protein 9 |
| A_44_P210440 | 2.17 | Akap13 | 293024 | A kinase (PRKA) anchor protein 13 |
| A_44_P1011464 | 2.17 | Rhobtb1 | 309722 | Rho-related BTB domain containing 1 |
| A_44_P116443 | 2.17 | Matr3 | 29150 | matrin 3 |
| A_44_P22150 | 2.16 | LOC684297 | 684297 | similar to PHD finger protein 8 |
| A_44_P402675 | 2.16 | Rxrb | 361801 | retinoid X receptor beta |
| A_44_P443542 | 2.15 | Mars2 | 316403 | methionyl-tRNA synthetase 2, mitochondrial |

TABLE 7

| ProbeName | RC | GeneSymbol | EntrezGene ID | GeneName |
|---|---|---|---|---|
| A_44_P391706 | 2.15 | Abcb8 | 362302 | ATP-binding cassette, sub-family B (MDR/TAP), member 8 |
| A_43_P12905 | 2.15 | Bik | 114496 | BCL2-interacting killer (apoptosis-inducing) |
| A_44_P652131 | 2.15 | Med22 | 499762 | mediator complex subunit 22 |
| A_44_P531758 | 2.14 | Cryab | 25420 | crystallin, alpha B |
| A_44_P357870 | 2.14 | Cpt1a | 25757 | carnitine palmitoyltransferase 1a, liver |
| A_42_P669388 | 2.14 | Cnksr1 | 298545 | connector enhancer of kinase suppressor of Ras 1 |
| A_44_P980916 | 2.14 | Kirrel3 | 315546 | kin of IRRE like 3 (Drosophila) |
| A_44_P698085 | 2.13 | Arl13a | 501617 | ADP-ribosylation factor-like 13A |
| A_44_P622113 | 2.13 | Sept8 | 303135 | septin 8 |
| A_44_P229079 | 2.12 | Rb1 | 24708 | retinoblastoma 1 |
| A_44_P258497 | 2.12 | Prmt2 | 499420 | protein arginine methyltransferase 2 |
| A_44_P422082 | 2.12 | Ccnb3 | 317389 | cyclin B3 |
| A_44_P823666 | 2.11 | Zhx2 | 314988 | zinc fingers and homeoboxes 2 |
| A_44_P991114 | 2.10 | Pds5b | 304218 | PDS5, regulator of cohesion maintenance, homolog B |
| | | | | (S. cerevisiae) |
| A_44_P180060 | 2.09 | Hdac6 | 84581 | histone deacetylase 6 |
| A_43_P13161 | 2.09 | Prdm4 | 170820 | PR domain containing 4 |
| A_44_P256345 | 2.08 | Smarcad1 | 312398 | SWI/SNF-related, matrix-associated actin-dependent |
| | | | | regulator of chromatin, subfamily a, containing DEAD/H box 1 |
| A_44_P398587 | 2.08 | Usp9x | 363445 | ubiquitin specific peptidase 9, X-linked |
| A_43_P14872 | 2.07 | Sdc1 | 25216 | syndecan 1 |
| A_44_P131290 | 2.07 | Arhgap28 | 301709 | Rho GTPase activating protein 28 |
| A_44_P208464 | 2.07 | Rad17 | 310034 | RAD17 homolog (S. pombe) |
| A_44_P996369 | 2.06 | Sorbs1 | 686098 | sorbin and SH3 domain containing 1 |
| A_43_P15281 | 2.06 | Tp63 | 246334 | tumor protein p63 |
| A_44_P410455 | 2.06 | Wdr48 | 363164 | WD repeat domain 48 |
| A_44_P148044 | 2.06 | Hnrpll | 313842 | heterogeneous nuclear ribonucleoprotein L-like |
| A_44_P837227 | 2.06 | LOC687994 | 687994 | similar to 82-kD FMRP Interacting Protein |
| A_44_P536282 | 2.06 | Sptbn1 | 305614 | spectrin, beta, non-erythrocytic 1 |
| A_44_P793692 | 2.05 | Dtx3l | 498089 | deltex 3-like (Drosophila) |
| A_44_P494439 | 2.05 | Cnnm2 | 294014 | cyclin M2 |
| A_44_P541053 | 2.04 | Fbln5 | 29158 | fibulin 5 |
| A_42_P815727 | 2.04 | Usp48 | 362636 | ubiquitin specific protease 48 |
| A_44_P1007347 | 2.03 | Tgm2 | 56083 | transglutaminase 2, C polypeptide |
| A_44_P304880 | 2.03 | RGD1564776 | 366473 | similar to ornithine decarboxylase-like protein |
| A_42_P678414 | 2.02 | RGD1563106 | 303238 | similar to novel protein |
| A_44_P191615 | 2.02 | Taok1 | 286993 | TAO kinase 1 |
| A_44_P473524 | 2.02 | Snx30 | 298033 | sorting nexin family member 30 |
| A_44_P147702 | 2.02 | Utp6 | 360574 | UTP6, small subunit (SSU) processome component, homolog |
| | | | | (yeast) |
| A_43_P23262 | 2.02 | Cytip | 311047 | cytohesin 1 interacting protein |
| A_43_P12682 | 2.02 | Rest | 83618 | RE1-silencing transcription factor |
| A_44_P279208 | 2.01 | Eif4g3 | 298573 | eukaryotic translation initiation factor 4 gamma, 3 |
| A_44_P1032103 | 2.01 | Adnp2 | 307236 | ADNP homeobox 2 |
| A_43_P22174 | 2.00 | Wdr1 | 360950 | WD repeat domain 1 |
| A_44_P575034 | 2.00 | Hiat1 | 100134827 | hippocampus abundant gene transcript 1 |

TABLE 8

| ProbeName | RC | GeneSymbol | EntrezGene ID | GeneName |
|---|---|---|---|---|
| A_44_P576954 | 2.00 | Ddx58 | 297989 | DEAD (Asp-Glu-Ala-Asp) box polypeptide 58 |
| A_44_P140332 | 2.00 | RGD1359600 | 307343 | LEA 4 domain containing protein RGD1359600 |
| A_43_P19525 | 2.00 | Arhgap12 | 307016 | Rho GTPase activating protein 12 |
| A_44_P574253 | 1.99 | Dapk2 | 300799 | death-associated kinase 2 |
| A_44_P824032 | 1.99 | Nup160 | 311182 | nucleoporin 160 |
| A_44_P702258 | 1.99 | Eea1 | 314764 | early endosome antigen 1 |
| A_43_P11363 | 1.98 | LOC363188 | 363188 | similar to ubiquitin protein ligase E3 component n-recognin 2 |
| A_44_P763063 | 1.98 | RGD1563437 | 291356 | similar to KIAA1217 |
| A_44_P638511 | 1.98 | Paqr5 | 315741 | progestin and adipoQ receptor family member V |
| A_44_P821966 | 1.97 | LOC500684 | 500684 | hypothetical protein LOC500684 |
| A_44_P306840 | 1.96 | Ddhd1 | 305816 | DDHD domain containing 1 |
| A_42_P806899 | 1.96 | Kdm6b | 363630 | lysine (K)-specific demethylase 6B |
| A_44_P834393 | 1.96 | Ube2d1 | 361831 | ubiquitin-conjugating enzyme E2D 1, UBC4/5 homolog (yeast) |
| A_44_P229501 | 1.96 | Rhoc | 295342 | ras homolog gene family, member C |
| A_44_P125042 | 1.96 | LOC361100 | 361100 | similar to topoisomerase (DNA) II beta |
| A_44_P116591 | 1.95 | Cx3cl1 | 89808 | chemokine (C-X3-C motif) ligand 1 |
| A_44_P178561 | 1.95 | LOC100302372 | 100302372 | hypothetical protein LOC100302372 |
| A_44_P335175 | 1.95 | RGD1311892 | 289399 | similar to hypothetical protein FLJ10901 |
| A_44_P543917 | 1.95 | Fbxo42 | 362646 | F-box protein 42 |
| A_42_P789482 | 1.95 | Daam1 | 314212 | dishevelled associated activator of morphogenesis 1 |
| A_44_P123033 | 1.94 | Klhl5 | 305351 | kelch-like 5 (Drosophila) |
| A_44_P177845 | 1.94 | Fbxo30 | 308283 | F-box protein 30 |
| A_44_P144188 | 1.94 | Syt12 | 191595 | synaptotagmin XII |
| A_42_P762924 | 1.94 | Herc1 | 315771 | hect (homologous to the E6-AP (UBE3A) carboxyl terminus) |
| | | | | domain and RCC1 (CHC1)-like domain (RLD) 1 |
| A_44_P249161 | 1.93 | Dab2ip | 192126 | DAB2 interacting protein |
| A_44_P466314 | 1.93 | Tacc2 | 309025 | transforming, acidic coiled-coil containing protein 2 |
| A_44_P1045515 | 1.93 | Sipa1l1 | 246212 | signal-induced proliferation-associated 1 like 1 |
| A_44_P267799 | 1.92 | Ets2 | 304063 | v-ets erythroblastosis virus E26 oncogene homolog 2 (avian) |
| A_44_P537673 | 1.92 | Zbtb43 | 311872 | zinc finger and BTB domain containing 43 |
| A_44_P250620 | 1.92 | Fhdc1 | 295161 | FH2 domain containing 1 |
| A_43_P12767 | 1.91 | Pdlim5 | 64353 | PDZ and LIM domain 5 |
| A_44_P163189 | 1.90 | Stxbp5 | 81022 | syntaxin binding protein 5 (tomosyn) |
| A_44_P669345 | 1.90 | Hdac8 | 363481 | histone deacetylase 8 |
| A_44_P450214 | 1.90 | Sestd1 | 295678 | SEC14 and spectrin domains 1 |
| A_42_P597638 | 1.90 | Acot2 | 192272 | acyl-CoA thioesterase 2 |
| A_44_P637827 | 1.89 | Fam117b | 363236 | family with sequence similarity 117, member B |
| A_44_P935860 | 1.88 | Nrip1 | 304157 | nuclear receptor interacting protein 1 |
| A_44_P265807 | 1.88 | Aldh3a2 | 65183 | aldehyde dehydrogenase 3 family, member A2 |
| A_43_P10919 | 1.88 | Exosc8 | 295050 | exosome component 8 |
| A_44_P508239 | 1.88 | Shmt1 | 287379 | serine hydroxymethyltransferase 1 (soluble) |
| A_44_P163816 | 1.88 | Lrrc57 | 311346 | leucine rich repeat containing 57 |
| A_44_P539012 | 1.87 | Rell2 | 361313 | RELT-like 2 |

TABLE 9

| ProbeName | RC | GeneSymbol | EntrezGene ID | GeneName |
|---|---|---|---|---|
| A_44_P699909 | 1.87 | Zfyve16 | 499508 | zinc finger, FYVE domain containing 16 |
| A_42_P762829 | 1.87 | Cebpd | 25695 | CCAAT/enhancer binding protein (C/EBP), delta |
| A_44_P484793 | 1.87 | Tnrc6b | 192178 | trinucleotide repeat containing 6B |
| A_44_P363549 | 1.87 | Nsun6 | 307148 | NOL1/NOP2/Sun domain family, member 6 |
| A_44_P744113 | 1.86 | Dennd5a | 308942 | DENN/MADD domain containing 5A |
| A_44_P335377 | 1.86 | Fbxw5 | 362081 | F-box and WD repeat domain containing 5 |
| A_42_P506402 | 1.86 | Slc25a25 | 246771 | solute carrier family 25 (mitochondrial carrier, phosphate |
| | | | | carrier), member 25 |
| A_44_P539035 | 1.86 | Dtwd1 | 296119 | DTW domain containing 1 |
| A_44_P992908 | 1.85 | Hspb1 | 24471 | heat shock protein 1 |
| A_42_P640641 | 1.84 | Homer2 | 29547 | homer homolog 2 (Drosophila) |
| A_44_P387324 | 1.83 | Nhej1 | 363251 | nonhomologous end-joining factor 1 |
| A_43_P10213 | 1.83 | Etnk1 | 312828 | ethanolamine kinase 1 |
| A_44_P314312 | 1.83 | Nol9 | 313744 | nucleolar protein 9 |
| A_42_P815566 | 1.82 | Med23 | 309565 | mediator complex subunit 23 |
| A_42_P511015 | 1.82 | Nup133 | 292085 | nucleoporin 133 |
| A_44_P562894 | 1.82 | Rnd1 | 362993 | Rho family GTPase 1 |
| A_42_P621736 | 1.81 | Rplp1 | 140661 | ribosomal protein, large, P1 |
| A_44_P576056 | 1.81 | Ankfy1 | 303292 | ankyrin repeat and FYVE domain containing 1 |
| A_44_P520829 | 1.81 | Pik3r4 | 363131 | phosphoinositide-3-kinase, regulatory subunit 4 |
| A_42_P586141 | 1.81 | Plcd1 | 24655 | phospholipase C, delta 1 |
| A_44_P337351 | 1.81 | Cxcl12 | 24772 | chemokine (C-X-C motif) ligand 12 (stromal cell-derived |
| | | | | factor 1) |
| A_44_P929844 | 1.80 | Lix1l | 499677 | Lix1 homolog (mouse)-like |
| A_44_P221056 | 1.80 | Ralbp1 | 84014 | ralA binding protein 1 |
| A_44_P315562 | 1.80 | Cald1 | 25687 | caldesmon 1 |
| A_44_P897591 | 1.79 | LOC302495 | 302495 | hypothetical LOC302495 |
| A_44_P561538 | 1.79 | Cdcp1 | 301082 | CUB domain containing protein 1 |
| A_44_P355758 | 1.79 | Ralb | 116546 | v-ral simian leukemia viral oncogene homolog B (ras related; |
| | | | | GTP binding protein) |
| A_44_P477116 | 1.78 | Pcyt1a | 140544 | phosphate cytidylyltransferase 1, choline, alpha |
| A_43_P20133 | 1.78 | Skil | 114208 | SKI-like oncogene |
| A_44_P318466 | 1.78 | Trim25 | 494338 | tripartite motif-containing 25 |
| A_44_P175875 | 1.78 | Pigv | 366478 | phosphatidylinositol glycan anchor biosynthesis, class V |
| A_44_P404198 | 1.77 | Mapk3 | 50689 | mitogen activated protein kinase 3 |
| A_43_P19455 | 1.77 | Pard6g | 307237 | par-6 partitioning defective 6 homolog gamma (C. elegans) |
| A_44_P439473 | 1.77 | Klf7 | 363243 | Kruppel-like factor 7 (ubiquitous) |
| A_44_P224259 | 1.77 | Pxn | 360820 | paxillin |
| A_44_P459918 | 1.76 | Dnaja4 | 300721 | DnaJ (Hsp40) homolog, subfamily A, member 4 |
| A_44_P454872 | 1.76 | Cnot1 | 291841 | CCR4-NOT transcription complex, subunit 1 |
| A_44_P313283 | 1.76 | Capn2 | 29154 | calpain 2 |
| A_43_P12384 | 1.76 | Birc3 | 78971 | baculoviral IAP repeat-containing 3 |
| A_44_P545945 | 1.76 | Frmd8 | 309172 | FERM domain containing 8 |
| A_44_P358627 | 1.76 | Chd2 | 308738 | chromodomain helicase DNA binding protein 2 |
| A_44_P850979 | 1.76 | Prpf39 | 314171 | PRP39 pre-mRNA processing factor 39 homolog (S. cerevisiae) |

TABLE 10

| ProbeName | RC | GeneSymbol | EntrezGene ID | GeneName |
|---|---|---|---|---|
| A_44_P173156 | 1.75 | Taf5 | 294018 | TAF5 RNA polymerase II, TATA box binding protein (TBP)- |
| | | | | associated factor |
| A_43_P19916 | 1.75 | Eif4enif1 | 305468 | eukaryotic translation initiation factor 4E nuclear import factor 1 |
| A_43_P15633 | 1.75 | Pkn2 | 207122 | protein kinase N2 |
| A_44_P639763 | 1.75 | Fam179b | 314169 | family with sequence similarity 179, member B |
| A_44_P929706 | 1.75 | Kdm2a | 361700 | lysine (K)-specific demethylase 2A |
| A_44_P402750 | 1.75 | Gda | 83585 | guanine deaminase |
| A_44_P914528 | 1.75 | Ccdc21 | 362622 | coiled-coil domain containing 21 |
| A_44_P151582 | 1.75 | Il17re | 362417 | interleukin 17 receptor E |
| A_42_P526712 | 1.74 | Eif4e3 | 297481 | eukaryotic translation initiation factor 4E member 3 |
| A_44_P920712 | 1.74 | Dock8 | 499337 | dedicator of cytokinesis 8 |
| A_44_P397763 | 1.74 | Nolc1 | 64896 | nucleolar and coiled-body phosphoprotein 1 |
| A_44_P372501 | 1.74 | Sult1a1 | 83783 | sulfotransferase family, cytosolic, 1A, phenol-preferring, |
| | | | | member 1 |
| A_44_P516818 | 1.74 | Ankrd17 | 289521 | ankyrin repeat domain 17 |
| A_44_P383360 | 1.73 | Trim2 | 361970 | tripartite motif-containing 2 |
| A_44_P466111 | 1.73 | Nfib | 29227 | nuclear factor I/B |
| A_44_P469584 | 1.73 | Cd14 | 60350 | CD14 molecule |
| A_43_P12519 | 1.73 | S100a10 | 81778 | S100 calcium binding protein A10 |
| A_44_P699502 | 1.73 | LOC687105 | 687105 | hypothetical protein LOC687105 |
| A_43_P23298 | 1.72 | Ccdc123 | 292811 | coiled-coil domain containing 123 |
| A_44_P226478 | 1.72 | RGD1309537 | 501203 | similar to Myosin regulatory light chain 2-A, smooth muscle |
| | | | | isoform (Myosin RLC-A) |
| A_44_P591548 | 1.72 | Pols | 306672 | polymerase (DNA directed) sigma |
| A_44_P472461 | 1.72 | Aplf | 500247 | aprataxin and PNKP like factor |
| A_44_P117270 | 1.72 | Gulp1 | 314543 | GULP, engulfment adaptor PTB domain containing 1 |
| A_44_P819401 | 1.72 | Slc40a1 | 170840 | solute carrier family 39 (iron-regulated transporter), member 1 |
| A_43_P20415 | 1.72 | Tfdp2 | 300947 | Transcription factor Dp-2 (E2F dimerization partner 2) |
| A_44_P1055082 | 1.71 | Dyrk2 | 314862 | dual-specificity tyrosine-(Y)-phosphorylation regulated kinase 2 |
| A_44_P472996 | 1.71 | Anxa5 | 25673 | annexin A5 |
| A_44_P1003794 | 1.71 | Nr3c1 | 24413 | nuclear receptor subfamily 3, group C, member 1 |
| A_42_P749968 | 1.71 | RGD1306862 | 287596 | similar to RIKEN cDNA 1200011M11 |
| A_44_P827926 | 1.71 | Rkhd2 | 307271 | ring finger and KH domain containing 2 |
| A_43_P11098 | 1.71 | Cdc42bpb | 113960 | CDC42 binding protein kinase beta (DMPK-like) |
| A_44_P100507 | 1.71 | Numa1 | 308870 | nuclear mitotic apparatus protein 1 |
| A_44_P335402 | 1.71 | Mocos | 361300 | molybdenum cofactor sulfurase |
| A_44_P1040642 | 1.70 | Mafb | 54264 | v-maf musculoaponeurotic fibrosarcoma oncogene homolog B |
| | | | | (avian) |
| A_44_P1033739 | 1.70 | Akap11 | 498549 | A kinase (PRKA) anchor protein 11 |
| A_44_P100009 | 1.70 | Diaph1 | 307483 | diaphanous homolog 1 (Drosophila) |
| A_44_P711020 | 1.70 | Znrf1 | 362367 | zinc and ring finger 1 |
| A_43_P14954 | 1.70 | Sos1 | 313845 | Son of sevenless homolog 1 (Drosophila) |
| A_43_P12245 | 1.70 | Rhob | 64373 | ras homolog gene family, member B |
| A_44_P319349 | 1.70 | Pkp2 | 287925 | plakophilin 2 |

TABLE 11

| ProbeName | RC | GeneSymbol | EntrezGene ID | GeneName |
|---|---|---|---|---|
| A_44_P143061 | 1.69 | Srf | 501099 | serum response factor (c-fos serum response element- |
| | | | | binding transcription factor) |
| A_44_P1007449 | 1.69 | RGD1310159 | 304500 | similar to acetyl-coA dehydrogenase -related (111.6 kD) |
| | | | | (5G231) |
| A_44_P142242 | 1.69 | Cdh1 | 83502 | cadherin 1 |
| A_44_P401284 | 1.69 | Rnf19a | 362900 | ring finger protein 19A |
| A_42_P802550 | 1.69 | Pdlim1 | 54133 | PDZ and LIM domain 1 |
| A_44_P105004 | 1.69 | Csnk1g3 | 64823 | casein kinase 1, gamma 3 |
| A_44_P164007 | 1.68 | Hectd1 | 362736 | HECT domain containing 1 |
| A_44_P338962 | 1.68 | Taf5l | 307927 | TAF5-like RNA polymerase II, p300/CBP-associated factor |
| | | | | (PCAF)-associated factor |
| A_43_P19059 | 1.68 | Noc3l | 361753 | nucleolar complex associated 3 homolog (S. cerevisiae) |
| A_44_P927671 | 1.68 | Nfat5 | 307820 | nuclear factor of activated T-cells 5 |
| A_44_P482953 | 1.68 | RGD1562529 | 311372 | similar to hypothetical protein FLJ21439 |
| A_44_P1033595 | 1.68 | Atl3 | 309187 | atlastin GTPase 3 |
| A_44_P244138 | 1.68 | Ptpn9 | 266611 | protein tyrosine phosphatase, non-receptor type 9 |
| A_44_P1034793 | 1.67 | Ncdd4l | 291553 | neural precursor cell expressed, developmentally down- |
| | | | | regulated 4-like |
| A_44_P345159 | 1.67 | Wipi1 | 303630 | WD repeat domain, phosphoinositide interacting 1 |
| A_44_P591729 | 1.67 | Gnal | 24611 | guanine nucleotide binding protein, alpha stimulating, olfactory |
| | | | | type |
| A_44_P246114 | 1.67 | Tle1 | 362533 | transducin-like enhancer of split 1 (E(sp1) homolog, |
| | | | | Drosophila) |
| A_44_P998638 | 1.67 | Pprc1 | 294007 | peroxisome proliferator-activated receptor gamma, |
| | | | | coactivator-related 1 |
| A_43_P12708 | 1.67 | Src | 83805 | v-src sarcoma (Schmidt-Ruppin A-2) viral oncogene homolog |
| | | | | (avian) |
| A_44_P465931 | 1.67 | Clic1 | 406864 | chloride intracellular channel 1 |
| A_43_P11255 | 1.67 | Secisbp2l | 296115 | SECIS binding protein 2-like |
| A_44_P1019407 | 1.67 | Rnf103 | 84508 | ring finger protein 103 |
| A_43_P21480 | 1.67 | Zfp772 | 308320 | zinc finger protein 772 |
| A_44_P956272 | 1.67 | Ogt | 26295 | O-linked N-acetylglucosamine (GlcNAc) transferase (UDP-N- |
| | | | | acetylglucosamine:polypeptide-N-acetylglucosaminyl transferase) |
| A_44_P653818 | 1.67 | Zcchc2 | 304695 | zinc finger, CCHC domain containing 2 |
| A_44_P393064 | 1.66 | Clasp1 | 304740 | cytoplasmic linker associated protein 1 |
| A_44_P398969 | 1.66 | Gtpbp2 | 363195 | GTP binding protein 2 |
| A_42_P538617 | 1.66 | Ift172 | 116475 | intraflagellar transport 172 homolog (Chlamydomonas) |
| A_44_P494768 | 1.66 | LOC500625 | 500625 | Hypothetical protein LOC500625 |
| A_42_P639047 | 1.66 | RGD1309350 | 293613 | similar to transthyretin (4L369) |
| A_44_P1037041 | 1.66 | Map3k6 | 313022 | mitogen-activated protein kinase kinase kinase 6 |
| A_44_P153774 | 1.66 | Plce1 | 114633 | phospholipase C, epsilon 1 |
| A_44_P229776 | 1.65 | Nedd4 | 25489 | neural precursor cell expressed, developmentally down- |
| | | | | regulated 4 |
| A_44_P1022889 | 1.65 | Cpne3 | 313087 | copine III |
| A_42_P524707 | 1.65 | Prim2 | 301323 | primase, DNA, polypeptide 2 |

TABLE 12

| ProbeName | RC | GeneSymbol | EntrezGene ID | GeneName |
|---|---|---|---|---|
| A_44_P545727 | 1.65 | Trafd1 | 114635 | TRAF type zinc finger domain containing 1 |
| A_44_P540542 | 1.64 | Myef2 | 679712 | myelin expression factor 2 |
| A_44_P297609 | 1.64 | Znf532 | 307362 | zinc finger protein 532 |
| A_44_P401170 | 1.64 | Tmem50b | 360698 | transmembrane protein 50B |
| A_42_P772157 | 1.64 | Il6ra | 24499 | interleukin 6 receptor, alpha |
| A_44_P930160 | 1.64 | Pdcd6ip | 501083 | programmed cell death 6 interacting protein |
| A_44_P447222 | 1.64 | LOC500757 | 500757 | similar to RalA binding protein 1 |
| A_43_P11514 | 1.63 | Tpm4 | 24852 | tropomyosin 4 |
| A_44_P445521 | 1.63 | Rnasen | 310159 | ribonuclease type III, nuclear |
| A_44_P466700 | 1.63 | Lpin1 | 313977 | lipin 1 |
| A_44_P1023498 | 1.63 | Flot1 | 64665 | flotillin 1 |
| A_42_P598531 | 1.63 | Cds1 | 81925 | CDP-diacylglycerol synthase 1 |
| A_44_P575991 | 1.63 | Tnfaip1 | 287543 | tumor necrosis factor, alpha-induced protein 1 (endothelial) |
| A_43_P22222 | 1.62 | Mudeng | 305861 | MU-2/AP1M2 domain containing, death-inducing |
| A_44_P383740 | 1.62 | Cdc2l5 | 306998 | cell division cycle 2-like 5 (cholinesterase-related cell division |
| | | | | controller) |
| A_44_P1004447 | 1.62 | Arid2 | 366980 | AT rich interactive domain 2 (Arid-rfx like) |
| A_44_P853624 | 1.62 | Znf654 | 288354 | zinc finger protein 654 |
| A_44_P351181 | 1.62 | Nat8b | 171084 | N-acetyltransferase 8B |
| A_44_P1013753 | 1.62 | Snx12 | 363478 | sorting nexin 12 |
| A_44_P680058 | 1.61 | Ap1g1 | 171494 | adaptor-related protein complex 1, gamma 1 subunit |
| A_44_P394042 | 1.61 | Itsn2 | 313934 | intersectin 2 |
| A_44_P388095 | 1.61 | Fam21c | 297530 | family with sequence similarity 21, member C |
| A_44_P532020 | 1.61 | RGD1306820 | 309069 | similar to erythroid differentiation-related factor 1 |
| A_42_P674856 | 1.61 | Mesdc1 | 308795 | mesoderm development candidate 1 |
| A_44_P419600 | 1.61 | Tmco6 | 291661 | transmembrane and coiled-coil domains 6 |
| A_44_P519167 | 1.61 | Fkbp5 | 361810 | FK506 binding protein 5 |
| A_42_P654627 | 1.60 | Prpf8 | 287530 | PRP8 pre-mRNA processing factor 8 homolog (S. cerevisiae) |
| A_44_P990043 | 1.60 | Pdcd11 | 309458 | programmed cell death 11 |
| A_44_P228942 | 1.60 | Runx1 | 50662 | runt-related transcription factor 1 |
| A_44_P622172 | 1.60 | Epn2 | 60443 | epsin 2 |
| A_43_P12803 | 1.60 | Arid4b | 84481 | AT rich interactive domain 4B (Rbp1 like) |
| A_44_P381906 | 1.60 | Slk | 54308 | STE20-like kinase (yeast) |
| A_42_P509385 | 1.60 | Prkx | 501563 | protein kinase, X-linked |
| A_44_P674198 | 1.59 | Dnajb3 | 680216 | DnaJ (Hsp40) homolog, subfamily B, member 3 |
| A_44_P181511 | 1.59 | Phf17 | 310352 | PHD finger protein 17 |
| A_44_P1036875 | 1.59 | Fam193a | 305452 | family with sequence similarity 193, member A |
| A_44_P290591 | 1.59 | Pvrl2 | 308417 | poliovirus receptor-related 2 |
| A_42_P544167 | 1.59 | Tep1 | 64523 | telomerase associated protein 1 |
| A_44_P895064 | 1.59 | Atxn7 | 361015 | ataxin 7 |
| A_44_P518127 | 1.58 | Wwtr1 | 295062 | WW domain containing transcription regulator 1 |
| A_44_P699644 | 1.58 | LOC313672 | 313672 | kazrin |
| A_43_P16935 | 1.58 | Mus81 | 293678 | MUS81 endonuclease homolog (S. cerevisiae) |
| A_44_P716856 | 1.58 | C1d | 289810 | nuclear DNA binding protein |
| A_44_P996338 | 1.58 | Arid4a | 314205 | AT rich interactive domain 4A (Rbp1 like) |

TABLE 13

| ProbeName | RC | GeneSymbol | EntrezGene ID | GeneName |
|---|---|---|---|---|
| A_44_P696669 | 1.58 | Ehd1 | 293692 | EH-domain containing 1 |
| A_44_P960156 | 1.58 | Usp32 | 303394 | ubiquitin specific protease 32 |
| A_44_P210178 | 1.58 | Carhsp1 | 260416 | calcium regulated heat stable protein 1 |
| A_44_P683893 | 1.57 | Acss1 | 296259 | acyl-CoA synthetase short-chain family member 1 |
| A_42_P630249 | 1.57 | Ctcf | 83726 | CCCTC-binding factor (zinc finger protein) |
| A_42_P772878 | 1.57 | Bag3 | 293524 | Bcl2-associated athanogene 3 |
| A_44_P333057 | 1.57 | Mtrr | 290947 | 5-methyltetrahydrofolate-homocysteine methyltransferase |
| | | | | reductase |
| A_42_P794901 | 1.57 | Ywhah | 25576 | tyrosine 3-monooxygenase/tryptophan 5-monooxygenase |
| | | | | activation protein, eta polypeptide |
| A_43_P11838 | 1.57 | Csrp1 | 29276 | cysteine and glycine-rich protein 1 |
| A_44_P421625 | 1.57 | Smc3 | 29486 | structural maintenance of chromosomes 3 |
| A_44_P158107 | 1.57 | Sorbs2 | 114901 | sorbin and SH3 domain containing 2 |
| A_44_P712484 | 1.57 | RGD1309762 | 304503 | similar to KIAA0614 protein |
| A_43_P11497 | 1.57 | Ptpn1 | 24697 | protein tyrosine phosphatase, non-receptor type 1 |
| A_44_P241583 | 1.56 | Setd2 | 316013 | SET domain containing 2 |
| A_44_P620665 | 1.56 | Efr3a | 362923 | EFR3 homolog A (S. cerevisiae) |
| A_44_P255905 | 1.56 | Tnk1 | 303247 | tyrosine kinase, non-receptor, 1 |
| A_44_P713311 | 1.56 | Ncor1 | 54299 | nuclear receptor co-repressor 1 |
| A_44_P144871 | 1.56 | Coq4 | 366013 | coenzyme Q4 homolog (S. cerevisiae) |
| A_44_P835214 | 1.56 | Klhl24 | 303803 | kelch-like 24 (Drosophila) |
| A_42_P678974 | 1.56 | Cox11 | 690300 | COX11 homolog, cytochrome c oxidase assembly protein (yeast) |
| A_44_P341428 | 1.55 | Tfip11 | 288718 | tuftelin interacting protein 11 |
| A_44_P456492 | 1.55 | Rcan1 | 266766 | regulator of calcineurin 1 |
| A_44_P269843 | 1.55 | Nagk | 297393 | N-acetylglucosamine kinase |
| A_44_P822616 | 1.55 | Ep300 | 170915 | E1A binding protein p300 |
| A_42_P761516 | 1.55 | Myst3 | 306571 | MYST histone acetyltransferase (monocytic leukemia) 3 |
| A_44_P387078 | 1.55 | Rbm28 | 312182 | RNA binding motif protein 28 |
| A_44_P142318 | 1.55 | Anapc7 | 304490 | anaphase promoting complex subunit 7 |
| A_44_P282605 | 1.55 | Ezh1 | 303547 | enhancer of zeste homolog 1 (Drosophila) |
| A_43_P15847 | 1.55 | Cyp27a1 | 301517 | cytochrome P450, family 27, subfamily a, polypeptide 1 |
| A_44_P684795 | 1.55 | Irs2 | 29376 | insulin receptor substrate 2 |
| A_44_P173935 | 1.55 | Sdccag1 | 100322884 | serologically defined colon cancer antigen 1 |
| A_44_P1025454 | 1.54 | Foxp1 | 297480 | forkhead box P1 |
| A_44_P670239 | 1.54 | Dusp18 | 305477 | dual specificity phosphatase 18 |
| A_44_P529743 | 1.54 | Hcca2 | 499288 | HCCA2 protein |
| A_44_P308308 | 1.54 | Fbxl8 | 498941 | F-box and leucine-rich repeat protein 8 |
| A_44_P639627 | 1.54 | Chd1 | 308215 | chromodomain helicase DNA binding protein 1 |
| A_44_P450019 | 1.54 | Tp53bp2 | 305025 | tumor protein p53 binding protein, 2″ |
| A_44_P1014086 | 1.54 | RGD1309995 | 314690 | similar to CG13957-PA |
| A_44_P516073 | 1.54 | RGD1310552 | 300836 | similar to hypothetical protein MGC38960 |
| A_44_P548468 | 1.53 | Kif5b | 117550 | kinesin family member 5B |
| A_43_P17164 | 1.53 | Pcaf | 301164 | p300/CBP-associated factor |
| A_44_P637482 | 1.53 | Slc25a42 | 689414 | solute carrier family 25, member 42 |
| A_43_P12552 | 1.53 | Ocln | 83497 | occludin |

TABLE 14

| ProbeName | RC | GeneSymbol | EntrezGene ID | GeneName |
|---|---|---|---|---|
| A_44_P277669 | 1.53 | RGD1559961 | 497974 | similar to novel protein |
| A_44_P197896 | 1.53 | Kifap3 | 289168 | kinesin-associated protein 3 |
| A_42_P797638 | 1.52 | Wtip | 361552 | Wilms tumor 1 interacting protein |
| A_43_P17071 | 1.52 | Gapvd1 | 311880 | GTPase activating protein and VPS9 domains 1 |
| A_44_P379805 | 1.52 | Ankrd42 | 293117 | ankyrin repeat domain 42 |
| A_44_P403978 | 1.52 | Supt4h1 | 287608 | suppressor of Ty 4 homolog 1 (S. cerevisiae) |
| A_44_P1007362 | 1.52 | Haus1 | 192228 | HAUS augmin-like complex, subunit 1 |
| A_44_P307106 | 1.52 | RGD1310951 | 313202 | similar to RIKEN cDNA E130308A19 |
| A_44_P529477 | 1.52 | Prdm2 | 313678 | PR domain containing 2, with ZNF domain |
| A_44_P618099 | 1.52 | Arvcf | 303798 | armadillo repeat gene deleted in velo-cardio-facial syndrome |
| A_44_P461085 | 1.52 | Gtf2h1 | 361580 | general transcription factor IIH, polypeptide 1 |
| A_44_P436040 | 1.51 | Rassf6 | 305251 | Ras association (RalGDS/AF-6) domain family member 6 |
| A_44_P142610 | 1.51 | Pcif1 | 362269 | PDX1 C-terminal inhibiting factor 1 |
| A_44_P885003 | 1.51 | Arap2 | 305367 | ArfGAP with RhoGAP domain, ankyrin repeat and PH domain 2 |
| A_44_P542785 | 1.51 | Ube2k | 289623 | ubiquitin-conjugating enzyme E2K (UBC1 homolog, yeast) |
| A_44_P558660 | 1.51 | Twf1 | 315265 | twinfilin, actin-binding protein, homolog 1 (Drosophila) |
| A_42_P774045 | 1.51 | Rpl8 | 26962 | ribosomal protein L8 |
| A_44_P398167 | 1.51 | Prdx1 | 117254 | peroxiredoxin 1 |
| A_44_P423600 | 1.51 | Phrf1 | 245925 | PHD and ring finger domains 1 |
| A_44_P188857 | 1.51 | Pcm1 | 81740 | pericentriolar material 1 |
| A_44_P486891 | 1.50 | Mapk12 | 60352 | mitogen-activated protein kinase 12 |

TABLE 15

| ProbeName | RC | GeneSymbol | EntrezGene ID | GeneName |
|---|---|---|---|---|
| A_44_P421391 | 0.03 | Slc1a4 | 305540 | solute carrier family 1 (glutamate/neutral amino acid |
| | | | | transporter), member 4 |
| A_44_P391196 | 0.11 | Prl3d4 | 24282 | prolactin family 3, subfamily d, member 4 |
| A_44_P251031 | 0.13 | Trim16 | 303214 | tripartite motif-containing 16 |
| A_44_P130916 | 0.13 | LOC689842 | 689842 | similar to Nucleolar GTP-binding protein 1 (Chronic renal |
| | | | | failure gene protein) (GTP-binding protein NGB) |
| A_44_P545717 | 0.13 | Cacna1e | 54234 | calcium channel, voltage-dependent, R type, alpha 1E subunit |
| A_44_P196401 | 0.14 | RT1-EC2 | 24737 | RT1 class Ib, locus EC2 |
| A_42_P614175 | 0.15 | Gal | 29141 | galanin prepropeptide |
| A_44_P540669 | 0.16 | Arnt2 | 25243 | aryl hydrocarbon receptor nuclear translocator 2 |
| A_44_P300215 | 0.17 | Stk11ip | 301535 | serine/threonine kinase 11 interacting protein |
| A_43_P12972 | 0.18 | Sdc3 | 116673 | syndecan 3 |
| A_44_P464237 | 0.18 | RGD1563155 | 367734 | similar to RIKEN cDNA 1700054O13 |
| A_44_P776231 | 0.19 | Tp53i11 | 311209 | tumor protein p53 inducible protein 11 |
| A_43_P11783 | 0.19 | Mpz | 24564 | myelin protein zero |
| A_44_P515281 | 0.19 | Dnmt3l | 309680 | DNA (cytosine-5-)-methyltransferase 3-like |
| A_44_P508355 | 0.19 | Ache | 83817 | acetylcholinesterase |
| A_44_P443101 | 0.19 | Cln5 | 306128 | ceroid-lipofuscinosis, neuronal 5 |
| A_44_P332545 | 0.20 | Olr184 | 293329 | olfactory receptor 184 |
| A_44_P409950 | 0.20 | Asb7 | 365277 | ankyrin repeat and SOCS box-containing 7 |
| A_44_P499528 | 0.20 | Rtp3 | 316018 | receptor (chemosensory) transporter protein 3 |
| A_42_P610733 | 0.20 | Peli2 | 305835 | pellino 2 |
| A_44_P363909 | 0.20 | Caskin2 | 303678 | cask-interacting protein 2 |
| A_44_P147396 | 0.21 | Cass4 | 296409 | Cas scaffolding protein family member 4 |
| A_44_P409835 | 0.22 | Plvap | 56765 | plasmalemma vesicle associated protein |
| A_42_P540609 | 0.22 | LOC680711 | 680711 | hypothetical protein LOC680711 |
| A_44_P172655 | 0.22 | Nat2 | 116632 | N-Acetyltransferase-2 (arylamine N-acetyltransferase) |
| A_44_P357111 | 0.22 | RGD1565498 | 500843 | similar to Hypothetical protein LOC270802 |
| A_44_P354078 | 0.22 | Hist1h1a | 291145 | histone cluster 1, H1a |
| A_44_P1037456 | 0.22 | Col22a1 | 315071 | collagen, type XXII, alpha 1 |
| A_44_P883270 | 0.23 | LOC100360528 | 100360528 | zinc finger-like protein-like |
| A_44_P114052 | 0.23 | Htr6 | 64354 | 5-hydroxytryptamine (serotonin) receptor 6 |
| A_44_P286439 | 0.23 | Trim42 | 301106 | tripartite motif-containing 42 |
| A_44_P112082 | 0.23 | Ccdc89 | 293107 | coiled-coil domain containing 89 |
| A_44_P326717 | 0.23 | Atp10d | 360932 | ATPase, class V, type 10D |
| A_44_P590906 | 0.23 | Klri2 | 503650 | killer cell lectin-like receptor family I member 2 |
| A_44_P130856 | 0.23 | LOC100360801 | 100360801 | mCG11385-like |
| A_44_P312038 | 0.23 | RGD1560019 | 367121 | similar to hypothetical protein |
| A_43_P15965 | 0.23 | Ugt2b37 | 29623 | UDP-glucuronosyltransferase 2 family, member 37 |
| A_43_P15269 | 0.23 | Oprk1 | 29335 | opioid receptor, kappa 1 |
| A_44_P493214 | 0.24 | Cyct | 25310 | cytochrome c, testis |
| A_44_P226928 | 0.24 | Pdzd2 | 65034 | PDZ domain containing 2 |
| A_44_P477830 | 0.24 | Vom2r31 | 286985 | vomeronasal 2 receptor, 31 |
| A_44_P370714 | 0.24 | Sh2d5 | 366489 | SH2 domain containing 5 |
| A_44_P999838 | 0.24 | Tmem136 | 315593 | transmembrane protein 136 |

TABLE 16

| ProbeName | RC | GeneSymbol | EntrezGene ID | GeneName |
|---|---|---|---|---|
| A_44_P172856 | 0.25 | Gh1 | 24391 | growth hormone 1 |
| A_44_P153753 | 0.25 | Ttl | 171572 | tubulin tyrosine ligase |
| A_44_P178179 | 0.25 | Pgbd5 | 292098 | piggyBac transposable element derived 5 |
| A_44_P728540 | 0.25 | Zfp583 | 499068 | zinc finger protein 583 |
| A_44_P262593 | 0.25 | Ankrd2 | 309374 | ankyrin repeat domain 2 (stretch responsive muscle) |
| A_42_P502621 | 0.26 | Avil | 79253 | advillin |
| A_44_P1003117 | 0.26 | Mcm3ap | 294339 | minichromosome maintenance complex component 3 |
| | | | | associated protein |
| A_44_P159259 | 0.26 | Mypn | 309760 | myopalladin |
| A_43_P20688 | 0.26 | Frmd4a | 307128 | FERM domain containing 4A |
| A_44_P856900 | 0.27 | Rnf182 | 498726 | ring finger protein 182 |
| A_44_P215575 | 0.27 | Kcng1 | 296395 | potassium voltage-gated channel, subfamily G, member 1 |
| A_43_P17340 | 0.27 | Paqr7 | 313615 | progestin and adipoQ receptor family member VII |
| A_44_P135531 | 0.27 | RGD1304929 | 290403 | similar to chromosome 13 open reading frame 18 |
| A_44_P232255 | 0.29 | RGD1565100 | 367308 | similar to alkaline phosphatase (EC 3.1.3.1) precursor, |
| | | | | placental-like - human |
| A_43_P20248 | 0.29 | Pkp1 | 304822 | plakophilin 1 |
| A_42_P577421 | 0.29 | Nnmt | 300691 | nicotinamide N-methyltransferase |
| A_44_P284129 | 0.29 | Csrnp1 | 363165 | cysteine-serine-rich nuclear protein 1 |
| A_43_P20745 | 0.29 | Siae | 363045 | sialic acid acetylesterase |
| A_44_P527123 | 0.29 | Hoxb6 | 497986 | homeo box B6 |
| A_44_P1001451 | 0.29 | Pga5 | 60372 | pepsinogen 5, group I |
| A_44_P436218 | 0.29 | Hoxd9 | 688999 | homeo box D9 |
| A_44_P517990 | 0.29 | RGD1308541 | 308768 | hypothetical LOC308768 |
| A_44_P299268 | 0.29 | Cyth3 | 116693 | cytohesin 3 |
| A_44_P171995 | 0.29 | Fbp1 | 24362 | fructose-1,6-bisphosphatase 1 |
| A_44_P885679 | 0.29 | Bnc1 | 365299 | basonuclin 1 |
| A_44_P1004324 | 0.29 | Col18a1 | 85251 | collagen, type XVIII, alpha 1 |
| A_44_P335636 | 0.30 | Traf3 | 362788 | Tnf receptor-associated factor 3 |
| A_44_P173554 | 0.30 | Sema7a | 315711 | sema domain, immunoglobulin domain (Ig), and GPI membrane |
| | | | | anchor, (semaphorin) 7A |
| A_44_P285726 | 0.31 | Rcn3 | 494125 | reticulocalbin 3, EF-hand calcium binding domain |
| A_44_P368220 | 0.31 | Ift122 | 312651 | intraflagellar transport 122 homolog (Chlamydomonas) |
| A_44_P344214 | 0.31 | Olr1275 | 405085 | olfactory receptor 1275 |
| A_44_P128986 | 0.31 | LOC619574 | 619574 | hypothetical protein LOC619574 |
| A_44_P534490 | 0.31 | Slc5a4a | 294341 | solute carrier family 5, member 4a |
| A_44_P323933 | 0.32 | Smc2 | 362519 | structural maintenance of chromosomes 2 |
| A_44_P621738 | 0.32 | Cldn18 | 315953 | claudin 18 |
| A_43_P17374 | 0.32 | LOC679462 | 679462 | similar to Tetraspanin-15 (Tspan-15) (Transmembrane 4 |
| | | | | superfamily member 15) (Tetraspan NET-7) |
| A_43_P13566 | 0.32 | Pogk | 304941 | pogo transposable element with KRAB domain |
| A_44_P346498 | 0.32 | Olr445 | 405936 | olfactory receptor 445 |
| A_44_P480954 | 0.32 | Abhd15 | 303343 | abhydrolase domain containing 15 |
| A_44_P110244 | 0.32 | LOC688219 | 688219 | similar to X-linked lymphocyte-regulated protein 3A |
| A_43_P20782 | 0.33 | RGD1564846 | 363885 | similar to RIKEN cDNA 2310015N07 |

TABLE 17

| ProbeName | RC | GeneSymbol | EntrezGene ID | GeneName |
|---|---|---|---|---|
| A_42_P556656 | 0.33 | Pias4 | 362827 | protein inhibitor of activated STAT, 4 |
| A_42_P650386 | 0.33 | Alg8 | 293129 | asparagine-linked glycosylation 8, alpha-1,3- |
| | | | | glucosyltransferase homolog (S. cerevisiae) |
| A_44_P445652 | 0.34 | Nkx2-4 | 366213 | NK2 homeobox 4 |
| A_44_P361809 | 0.34 | Mmp2 | 81686 | matrix metallopeptidase 2 |
| A_44_P744940 | 0.34 | Iqcf1 | 503227 | IQ motif containing F1 |
| A_44_P281154 | 0.34 | B9d2 | 308443 | B9 protein domain 2 |
| A_44_P215393 | 0.34 | Fam107a | 361018 | family with sequence similarity 107, member A |
| A_44_P199096 | 0.34 | Rarres1 | 310486 | retinoic acid receptor responder (tazarotene induced) 1 |
| A_44_P351837 | 0.35 | RGD1561276 | 499857 | similar to olfactory receptor Olfr1289 |
| A_44_P342117 | 0.35 | Wbp2 | 192645 | WW domain binding protein 2 |
| A_44_P698187 | 0.35 | Wdr93 | 499189 | WD repeat domain 93 |
| A_42_P615374 | 0.35 | C1qtnf5 | 315598 | C1q and tumor necrosis factor related protein 5 |
| A_44_P104756 | 0.36 | Olr1303 | 405080 | olfactory receptor 1303 |
| A_42_P799113 | 0.36 | Egfl7 | 245963 | EGF-like-domain, multiple 7 |
| A_44_P383975 | 0.36 | Olr507 | 404881 | olfactory receptor 507 |
| A_42_P635881 | 0.36 | Slc32a1 | 83612 | solute carrier family 32 (GABA vesicular transporter), member 1 |
| A_44_P262670 | 0.36 | RGD1563516 | 690148 | similar to histone protein Hist2h3c1 |
| A_44_P139744 | 0.36 | Id3 | 25585 | inhibitor of DNA binding 3 |
| A_44_P393778 | 0.36 | LOC691130 | 691130 | hypothetical protein LOC691130 |
| A_42_P714281 | 0.36 | Ccdc58 | 288065 | coiled-coil domain containing 58 |
| A_44_P470992 | 0.36 | Sox21 | 306168 | SRY (sex determining region Y)-box 21 |
| A_42_P469248 | 0.36 | Tnfsf12 | 360548 | tumor necrosis factor ligand superfamily member 12 |
| A_42_P719350 | 0.36 | Grich2 | 501747 | glutamine rich 2 |
| A_44_P182835 | 0.36 | RGD1308930 | 291069 | similar to Protein phosphatase 1, regulatory subunit 3D |
| | | | | (Protein phosphatase 1, regulatory subunit 6) (Protein |
| | | | | phosphatase 1 binding subunit R6) |
| A_44_P316185 | 0.36 | LOC307727 | 307727 | similar to Probable chromodomain-helicase-DNA-binding |
| | | | | protein KIAA1416 |
| A_43_P23467 | 0.36 | B3galnt2 | 291212 | beta-1,3-N-acetylgalactosaminyltransferase 2 |
| A_43_P18043 | 0.37 | Tob2 | 315159 | transducer of ERBB2, 2 |
| A_42_P600372 | 0.37 | Creb3l1 | 362165 | cAMP responsive element binding protein 3-like 1 |
| A_43_P23458 | 0.37 | Klk12 | 308564 | kallikrein related-peptidase 12 |
| A_44_P1013248 | 0.37 | Dqx1 | 680434 | DEAQ box RNA-dependent ATPase 1 |
| A_43_P12695 | 0.37 | Ncoa2 | 83724 | nuclear receptor coactivator 2 |
| A_44_P989558 | 0.37 | Slfnl1 | 500540 | schlafen-like 1 |
| A_42_P730411 | 0.37 | Elf2 | 361944 | E74-like factor 2 |
| A_43_P19268 | 0.37 | Gsx2 | 364140 | GS homeobox 2 |
| A_43_P18612 | 0.37 | Hhip | 291936 | Hedgehog-interacting protein |
| A_43_P15751 | 0.37 | Grm2 | 24415 | glutamate receptor, metabotropic 2 |
| A_44_P728665 | 0.37 | Fam170a | 498864 | family with sequence similarity 170, member A |
| A_44_P403302 | 0.37 | Pnma3 | 293840 | paraneoplastic antigen MA3 |
| A_42_P685404 | 0.38 | Gpc2 | 171517 | glypican 2 |
| A_42_P651681 | 0.38 | Npvf | 60570 | neuropeptide VF precursor |
| A_44_P376505 | 0.38 | RGD1308143 | 316685 | similar to D330021B20 protein |

TABLE 18

| ProbeName | RC | GeneSymbol | EntrezGene ID | GeneName |
|---|---|---|---|---|
| A_44_P999653 | 0.38 | Tsen54 | 690308 | tRNA splicing endonuclease 54 homolog (S. cerevisiae) |
| A_44_P422836 | 0.38 | Igfbp1 | 25685 | insulin-like growth factor binding protein 1 |
| A_44_P197213 | 0.39 | Zdhhc19 | 288045 | zinc finger, DHHC-type containing 19 |
| A_43_P17917 | 0.39 | Csrnp2 | 315308 | cysteine-serine-rich nuclear protein 2 |
| A_44_P119244 | 0.39 | Dcst2 | 295247 | DC-STAMP domain containing 2 |
| A_44_P405022 | 0.39 | Krt1 | 300250 | keratin 1 |
| A_44_P389216 | 0.39 | Nanos1 | 365494 | nanos homolog 1 (Drosophila) |
| A_42_P749820 | 0.39 | Cdh13 | 192248 | cadherin 13 |
| A_44_P1020757 | 0.40 | Ebi3 | 680609 | Epstein-Barr virus induced 3 |
| A_44_P299349 | 0.40 | Prr3 | 361788 | proline rich 3 |
| A_43_P23147 | 0.40 | Rnf43 | 303412 | ring finger protein 43 |
| A_44_P291196 | 0.40 | Btbd17 | 303660 | BTB (POZ) domain containing 17 |
| A_44_P402594 | 0.40 | Olr1626 | 290002 | olfactory receptor 1626 |
| A_42_P589355 | 0.41 | Trim26 | 309586 | tripartite motif-containing 26 |
| A_44_P383310 | 0.41 | Gata3 | 85471 | GATA binding protein 3 |
| A_44_P374620 | 0.41 | Cdkn1c | 246060 | cyclin-dependent kinase inhibitor 1C |
| A_44_P353405 | 0.41 | Hrc | 292905 | histidine rich calcium binding protein |
| A_43_P13326 | 0.41 | Grasp | 192254 | GRP1 (general receptor for phosphoinositides 1)-associated |
| | | | | scaffold protein |
| A_44_P546279 | 0.42 | Galm | 313843 | galactose mutarotase (aldose 1-epimerase) |
| A_44_P138388 | 0.42 | RGD1308106 | 361719 | LOC361719 |
| A_44_P895110 | 0.42 | Samd14 | 287637 | sterile alpha motif domain containing 14 |
| A_44_P456584 | 0.42 | Olr471 | 406046 | olfactory receptor 471 |
| A_44_P172990 | 0.42 | Oprd1 | 24613 | opioid receptor, delta 1 |
| A_43_P11786 | 0.43 | Pim1 | 24649 | pim-1 oncogene |
| A_44_P265709 | 0.43 | Il2rb | 25746 | interleukin 2 receptor, beta |
| A_43_P17652 | 0.43 | Ptpn13 | 498331 | protein tyrosine phosphatase, non-receptor type 13 |
| A_43_P14440 | 0.43 | Mtus1 | 306487 | mitochondrial tumor suppressor 1 |
| A_44_P161723 | 0.43 | RGD1305298 | 299330 | hypothetical LOC299330 |
| A_44_P363736 | 0.43 | Fhl3 | 313582 | four and a half LIM domains 3 |
| A_44_P278529 | 0.43 | Olr515 | 295768 | olfactory receptor 515 |
| A_43_P12022 | 0.43 | Tpsab1 | 54271 | tryptase alpha/beta 1 |
| A_44_P264965 | 0.44 | RGD1564961 | 294622 | similar to retinoblastoma-binding protein 1 isoform I |
| A_44_P152120 | 0.44 | Hdhd2 | 361351 | haloacid dehalogenase-like hydrolase domain containing 2 |
| A_44_P257832 | 0.44 | Farp1 | 306183 | FERM, RhoGEF (Arhgef) and pleckstrin domain protein 1 |
| | | | | (chondrocyte-derived) |
| A_44_P542390 | 0.44 | Ptprs | 25529 | protein tyrosine phosphatase, receptor type, S |
| A_42_P574345 | 0.45 | Hand1 | 59112 | heart and neural crest derivatives expressed 1 |
| A_44_P179210 | 0.45 | Rpl37a | 363248 | ribosomal protein L37a |
| A_44_P124013 | 0.45 | Tcf15 | 296272 | transcription factor 15 |
| A_44_P458901 | 0.45 | Neurod2 | 54276 | neurogenic differentiation 2 |
| A_43_P17714 | 0.45 | Usp20 | 311856 | ubiquitin specific peptidase 20 |
| A_43_P16998 | 0.45 | Foxo3 | 294515 | forkhead box O3 |
| A_44_P506064 | 0.45 | Aldh1l2 | 299699 | aldehyde dehydrogenase 1 family, member L2 |
| A_44_P383199 | 0.45 | RGD1308706 | 291925 | similar to RIKEN cDNA 4921524J17 |

TABLE 19

| ProbeName | RC | GeneSymbol | EntrezGene ID | GeneName |
|---|---|---|---|---|
| A_44_P503251 | 0.45 | Nov | 81526 | nephroblastoma overexpressed gene |
| A_42_P685320 | 0.46 | RGD1566130 | 312504 | similar to mKIAA1940 protein |
| A_44_P522580 | 0.46 | Slc34a2 | 84395 | solute carrier family 34 (sodium phosphate), member 2 |
| A_42_P663871 | 0.46 | Gzma | 266708 | granzyme A |
| A_43_P15563 | 0.46 | Hist1h4b | 64627 | histone cluster 1, H4b |
| A_44_P482880 | 0.46 | Iqgap3 | 310621 | IQ motif containing GTPase activating protein 3 |
| A_44_P341075 | 0.47 | Enpp2 | 84050 | ectonucleotide pyrophosphatase/phosphodiesterase 2 |
| A_42_P814765 | 0.47 | Tm7sf2 | 293688 | transmembrane 7 superfamily member 2 |
| A_43_P22672 | 0.47 | Exo1 | 305000 | exonuclease 1 |
| A_42_P548840 | 0.47 | Slc9a1 | 24782 | solute carrier family 9 (sodium/hydrogen exchanger), member 1 |
| A_44_P137047 | 0.47 | Rpl37 | 81770 | ribosomal protein L37 |
| A_44_P154298 | 0.47 | RGD1565970 | 498519 | similar to mast cell protease 8 |
| A_44_P440392 | 0.47 | Pde12 | 306231 | phosphodiesterase 12 |
| A_44_P983152 | 0.47 | Eefsec | 500255 | eukaryotic elongation factor, selenocysteine-tRNA-specific |
| A_44_P250879 | 0.47 | Dmrta2 | 313471 | DMRT-like family A2 |
| A_44_P377515 | 0.48 | LOC498685 | 498685 | similar to UPF0308 protein C9orf21 |
| A_43_P21008 | 0.48 | Brunol5 | 314647 | bruno-like 5, RNA binding protein (Drosophila) |
| A_43_P15859 | 0.48 | Ocm | 25503 | oncomodulin |
| A_44_P100139 | 0.49 | Olr4 | 292560 | olfactory receptor 4 |
| A_42_P680186 | 0.49 | Tmem164 | 367763 | transmembrane protein 164 |
| A_44_P477954 | 0.49 | RGD1561843 | 308263 | similar to 60S ribosomal protein L23a |
| A_44_P508711 | 0.49 | Flad1 | 751787 | flavin adenine dinucleotide synthetase |
| A_44_P543303 | 0.49 | Frmd4b | 252858 | FERM domain containing 4B |
| A_42_P808973 | 0.49 | Marcksl1 | 81520 | MARCKS-like 1 |
| A_44_P506258 | 0.49 | Bhlhe22 | 365748 | basic helix-loop-helix family, member e22 |
| A_44_P430803 | 0.50 | Yif1b | 292768 | Yip1 interacting factor homolog B (S. cerevisiae) |
| A_44_P340434 | 0.50 | Ttc38 | 300125 | tetratricopeptide repeat domain 38 |
| A_44_P191022 | 0.51 | Phyh | 114209 | phytanoyl-CoA 2-hydroxylase |
| A_44_P504244 | 0.51 | Mfsd6 | 301388 | major facilitator superfamily domain containing 6 |
| A_44_P605454 | 0.51 | LOC257642 | 257642 | rRNA promoter binding protein |
| A_42_P654862 | 0.52 | Fam189b | 310640 | family with sequence similarity 189, member B |
| A_44_P218263 | 0.52 | Sned1 | 316638 | sushi, nidogen and EGF-like domains 1 |
| A_44_P456836 | 0.52 | LOC100362849 | 100362849 | hypothetical protein LOC100362849 |
| A_42_P579174 | 0.52 | Cd3d | 25710 | CD3 molecule delta polypeptide |
| A_43_P20593 | 0.53 | Slc25a29 | 314441 | solute carrier family 25, member 29 |
| A_43_P18361 | 0.53 | Cd7 | 303747 | Cd7 molecule |
| A_44_P131296 | 0.53 | RGD1563261 | 287668 | similar to leucine-rich repeat domain-containing protein |
| A_43_P12795 | 0.53 | Grb7 | 84427 | growth factor receptor bound protein 7 |
| A_44_P798959 | 0.53 | Ccdc147 | 309467 | coiled-coil domain containing 147 |
| A_44_P487798 | 0.53 | Stat2 | 288774 | signal transducer and activator of transcription 2 |
| A_42_P643349 | 0.53 | LOC688228 | 688228 | similar to Myosin light polypeptide 4 (Myosin light chain 1, |
| | | | | atrial isoform) |

TABLE 20

| ProbeName | RC | GeneSymbol | EntrezGene ID | GeneName |
|---|---|---|---|---|
| A_44_P221588 | 0.53 | Ppp3ca | 24674 | protein phosphatase 3 (formerly 2B), catalytic subunit, alpha |
| | | | | isoform |
| A_43_P19262 | 0.53 | LOC688241 | 688241 | Ssimilar to GTPase activating protein testicular GAP1 |
| A_44_P548257 | 0.54 | Shisa4 | 360848 | shisa homolog 4 (Xenopus laevis) |
| A_44_P1000391 | 0.54 | Atp8b1 | 291555 | ATPase, Class I, type 8B, member 1 |
| A_44_P156521 | 0.54 | Krtap1-5 | 497995 | keratin associated protein 1-5 |
| A_42_P598602 | 0.54 | Cd3g | 300678 | CD3 molecule, gamma polypeptide |
| A_44_P210907 | 0.54 | Txnl4b | 292008 | thioredoxin-like 4B |
| A_44_P825558 | 0.54 | Ranbp10 | 361396 | RAN binding protein 10 |
| A_44_P1039760 | 0.54 | Dynlt3 | 363448 | dynein light chain Tctex-type 3 |
| A_44_P535337 | 0.55 | Atp2b2 | 24215 | ATPase, Ca++ transporting, plasma membrane 2 |
| A_44_P403005 | 0.55 | Trub1 | 361775 | TruB pseudouridine (psi) synthase homolog 1 (E. coli) |
| A_44_P327997 | 0.55 | Zhx3 | 311604 | zinc fingers and homeoboxes 3 |
| A_44_P227228 | 0.55 | Rexo4 | 311826 | REX4, RNA exonuclease 4 homolog (S. cerevisiae) |
| A_44_P231628 | 0.55 | Snapc2 | 304204 | small nuclear RNA activating complex, polypeptide 2 |
| A_44_P888303 | 0.56 | LOC685031 | 685031 | hypothetical protein LOC685031 |
| A_43_P22864 | 0.56 | Rad54l | 298429 | RAD54 like (S. cerevisiae) |
| A_44_P365694 | 0.56 | Vps37c | 308178 | vacuolar protein sorting 37 homolog C (S. cerevisiae) |
| A_44_P249033 | 0.56 | Hmga1 | 117062 | high mobility group AT-hook 1 |
| A_44_P299641 | 0.56 | Olr1572 | 286490 | olfactory receptor 1572 |
| A_44_P240953 | 0.56 | Furin | 54281 | furin (paired basic amino acid cleaving enzyme) |
| A_42_P511274 | 0.56 | RT1-CE5 | 309607 | RT1 class I, locus CE5 |
| A_44_P186336 | 0.56 | Rab15 | 299156 | RAB15, member RAS oncogene family |
| A_43_P13199 | 0.56 | Folr1 | 171049 | folate receptor 1 (adult) |
| A_44_P301420 | 0.56 | Fundc1 | 363442 | FUN14 domain containing 1 |
| A_44_P208304 | 0.56 | Pak3 | 29433 | p21 protein (Cdc42/Rac)-activated kinase 3 |
| A_42_P763077 | 0.57 | Aqp2 | 25386 | aquaporin 2 (collecting duct) |
| A_44_P203355 | 0.57 | Hapln2 | 64057 | hyaluronan and proteoglycan link protein 2 |
| A_43_P13363 | 0.57 | Tmem37 | 245953 | transmembrane protein 37 |
| A_44_P199541 | 0.57 | Gjd3 | 363677 | gap junction protein, delta 3 |
| A_43_P18993 | 0.57 | Lamb1 | 298941 | laminin, beta 1 |
| A_44_P474660 | 0.57 | Cox5a | 252934 | cytochrome c oxidase, subunit Va |
| A_43_P15750 | 0.57 | Cfd | 54249 | complement factor D (adipsin) |
| A_42_P492542 | 0.57 | Jak3 | 25326 | Janus kinase 3 |
| A_44_P191593 | 0.57 | Magt1 | 116967 | magnesium transporter 1 |
| A_43_P13088 | 0.57 | Sc4mol | 140910 | sterol-C4-methyl oxidase-like |
| A_44_P1034637 | 0.58 | Uros | 309070 | uroporphyrinogen III synthase |
| A_43_P11924 | 0.58 | Calca | 24241 | calcitonin-related polypeptide alpha |
| A_44_P186951 | 0.58 | Abca7 | 299609 | ATP-binding cassette, sub-family A (ABC1), member 7 |
| A_44_P221295 | 0.58 | Pm20d2 | 313130 | peptidase M20 domain containing 2 |
| A_44_P252946 | 0.58 | Dpf2 | 361711 | D4, zinc and double PHD fingers family 2 |
| A_44_P352079 | 0.58 | Tmprss4 | 367074 | transmembrane protease, serine 4 |
| A_44_P638290 | 0.58 | Znf467 | 500110 | zinc finger protein 467 |
| A_44_P323882 | 0.58 | Btbd9 | 294318 | BTB (POZ) domain containing 9 |

TABLE 21

| ProbeName | RC | GeneSymbol | EntrezGene ID | GeneName |
|---|---|---|---|---|
| A_44_P226832 | 0.58 | Rasgrf1 | 192213 | RAS protein-specific guanine nucleotide-releasing factor 1 |
| A_44_P197104 | 0.58 | RGD1310270 | 314798 | similar to CG33154-PB |
| A_42_P534068 | 0.58 | Adam15 | 57025 | a disintegrin and metallopeptidase domain 15 (metargidin) |
| A_44_P388755 | 0.58 | Kif15 | 353302 | kinesin family member 15 |
| A_44_P530187 | 0.59 | Plekho2 | 315764 | pleckstrin homology domain containing, family O member 2 |
| A_43_P13278 | 0.59 | Atp2c2 | 171496 | ATPase, Ca++ transporting, type 2C, member 2 |
| A_44_P967625 | 0.59 | Rps25 | 122799 | ribosomal protein s25 |
| A_44_P306368 | 0.59 | Lipa | 25055 | lipase A, lysosomal acid, cholesterol esterase |
| A_42_P643698 | 0.59 | Cpeb2 | 360949 | cytoplasmic polyadenylation element binding protein 2 |
| A_42_P502363 | 0.59 | Znf575 | 308430 | zinc finger protein 575 |
| A_44_P257528 | 0.60 | LOC287010 | 287010 | thiol-specific antioxidant-like protein |
| A_44_P363585 | 0.60 | Arhgap26 | 307459 | Rho GTPase activating protein 26 |
| A_44_P464169 | 0.60 | LOC311352 | 311352 | similar to Adenosine deaminase CG11994-PA |
| A_44_P333266 | 0.60 | Znrf4 | 301127 | zinc and ring finger 4 |
| A_43_P23342 | 0.60 | Msh5 | 294252 | mutS homolog 5 (E. coli) |
| A_44_P998423 | 0.60 | Tmed7 | 252889 | transmembrane emp24 protein transport domain containing 7 |
| A_43_P20024 | 0.60 | Gpd1l | 363159 | glycerol-3-phosphate dehydrogenase 1-like |
| A_42_P664913 | 0.60 | Ankrd37 | 361149 | ankyrin repeat domain 37 |
| A_43_P12813 | 0.60 | Cox4i2 | 84683 | cytochrome c oxidase subunit IV isoform 2 |
| A_44_P541438 | 0.60 | Msl3l2 | 309790 | male-specific lethal 3-like 2 (Drosophila) |
| A_43_P20085 | 0.61 | Snrnp70 | 361574 | small nuclear ribonucleoprotein 70 (U1) |
| A_43_P21439 | 0.61 | Rps6ka5 | 314384 | ribosomal protein S6 kinase, polypeptide 5 |
| A_44_P232851 | 0.61 | Tomm20 | 266601 | translocase of outer mitochondrial membrane 20 homolog |
| | | | | (yeast) |
| A_44_P314219 | 0.61 | Rassf4 | 362423 | Ras association (RalGDS/AF-6) domain family member 4 |
| A_44_P435739 | 0.61 | Hes1 | 29577 | hairy and enhancer of split 1 (Drosophila) |
| A_44_P368406 | 0.61 | Ctdsp1 | 363249 | CTD (carboxy-terminal domain, RNA polymerase II, |
| | | | | polypeptide A) small phosphatase 1 |
| A_43_P13381 | 0.61 | Pomc | 24664 | proopiomelanocortin |
| A_44_P203411 | 0.61 | Inpp4b | 116699 | inositol polyphosphate-4-phosphatase, type II |
| A_44_P515410 | 0.62 | Nfix | 81524 | nuclear factor I/X (CCAAT-binding transcription factor) |
| A_44_P239306 | 0.62 | Ydjc | 287938 | YdjC homolog (bacterial) |
| A_44_P110109 | 0.62 | Cst3 | 25307 | cystatin C |
| A_44_P313815 | 0.62 | Tesk2 | 170908 | testis-specific kinase 2 |
| A_44_P544351 | 0.62 | Tcn2 | 64365 | transcobalamin 2 |
| A_44_P217726 | 0.62 | RGD1309847 | 308894 | similar to peptidylglycine alpha-amidating monooxygenase |
| | | | | COOH-terminal interactor; peptidylglycine alpha-amidating |
| | | | | monooxygenase COOH-terminal interactor protein-1 |
| A_44_P163369 | 0.63 | Artn | 362572 | artemin |
| A_42_P505188 | 0.63 | Wibg | 366790 | within bgcn homolog (Drosophila) |

TABLE 22

| ProbeName | RC | GeneSymbol | EntrezGene ID | GeneName |
|---|---|---|---|---|
| A_44_P330931 | 0.63 | Prpf4 | 298095 | PRP4 pre-mRNA processing factor 4 homolog (yeast) |
| A_44_P107972 | 0.63 | LOC685179 | 685179 | similar to SWI/SNF-related matrix-associated actin- |
| | | | | dependent regulator of chromatin c2 |
| A_44_P868418 | 0.63 | Igsf3 | 295325 | immunoglobulin superfamily, member 3 |
| A_44_P388973 | 0.63 | Got1 | 24401 | glutamic-oxaloacetic transaminase 1, soluble (aspartate |
| | | | | aminotransferase 1) |
| A_43_P13117 | 0.63 | Lcp2 | 155918 | lymphocyte cytosolic protein 2 |
| A_44_P416633 | 0.63 | Agrn | 25592 | agrin |
| A_44_P307980 | 0.63 | Cited2 | 114490 | Cbp/p300-interacting transactivator, with Glu/Asp-rich |
| | | | | carboxy-terminal domain, 2 |
| A_44_P381781 | 0.64 | Bat4 | 415064 | HLA-B associated transcript 4 |
| A_44_P152237 | 0.64 | RGD1308059 | 362535 | similar to DNA segment, Chr 4, Brigham & Womens Genetics |
| | | | | 0951 expressed |
| A_42_P819377 | 0.64 | Sft2d1 | 292305 | SFT2 domain containing 1 |
| A_44_P199077 | 0.64 | Ubac2 | 361094 | UBA domain containing 2 |
| A_44_P105697 | 0.64 | Scly | 363285 | selenocysteine lyase |
| A_44_P100023 | 0.64 | Wrb | 288233 | tryptophan rich basic protein |
| A_44_P139686 | 0.64 | Zfp161 | 282825 | zinc finger protein 161 |
| A_44_P370122 | 0.64 | Fads2 | 83512 | fatty acid desaturase 2 |
| A_44_P370426 | 0.64 | Fbxo8 | 306436 | F-box protein 8 |
| A_44_P391750 | 0.64 | RGD1563148 | 362447 | similar to osteoclast inhibitory lectin |
| A_43_P16059 | 0.64 | Vegfb | 89811 | vascular endothelial growth factor B |
| A_44_P1016480 | 0.65 | Lpl | 24539 | lipoprotein lipase |
| A_44_P112428 | 0.65 | Chaf1a | 363333 | chromatin assembly factor 1, subunit A (p150) |
| A_44_P997787 | 0.65 | Srrd | 288717 | SRR1 domain containing |
| A_44_P1013006 | 0.65 | Steap1 | 297738 | six transmembrane epithelial antigen of the prostate 1 |
| A_44_P192543 | 0.65 | Fkbp11 | 300211 | FK506 binding protein 11 |
| A_42_P698051 | 0.65 | Slc39a3 | 314637 | solute carrier family 39 (zinc transporter), member 3 |
| A_44_P271842 | 0.65 | Zfp53 | 308236 | zinc finger protein 53 |
| A_44_P372251 | 0.65 | Bcan | 25393 | brevican |
| A_44_P633261 | 0.65 | Foxc2 | 171356 | forkhead box C2 |
| A_43_P23135 | 0.65 | Jph1 | 297748 | junctophilin 1 |
| A_42_P683840 | 0.65 | Ccl27 | 362505 | chemokine (C-C motif) ligand 27 |
| A_44_P189326 | 0.65 | Pdia3 | 29468 | protein disulfide isomerase family A, member 3 |
| A_43_P15862 | 0.66 | Gabrq | 65187 | gamma-aminobutyric acid (GABA) receptor, theta |
| A_44_P358807 | 0.66 | Surf6 | 303076 | surfeit 6 |
| A_43_P13119 | 0.66 | Runx3 | 156726 | runt-related transcription factor 3 |
| A_44_P446882 | 0.66 | Abcb6 | 140669 | ATP-binding cassette, sub-family B (MDR/TAP), member 6 |
| A_42_P785419 | 0.67 | Cdk2 | 362817 | cyclin dependent kinase 2 |
| A_44_P863933 | 0.67 | LOC687813 | 687813 | similar to Tnf receptor-associated factor 1 |
| A_43_P19071 | 0.67 | Porcn | 317368 | porcupine homolog (Drosophila) |

The mRNAs of which expression amounts increased included those derived from genes coding for cytokine receptors such as Il6ra and Il-17re, which are receptors of IL-6 and IL-17 playing important roles in defense against infection, and genes coding for chemokines such as Cxcl2 and Cxcl12, which induce migration of neutrophils to function for the defense against infection, and Cx3c11, which induce migration of cells expressing CX3CR1 (monocytes, NK cells, T cells, etc.) to function for the defense against infection. It is considered that increase in expression of these genes contributes to immunostimulation.

Further, the mRNAs of which expression amounts increased also included those derived for genes coding for such proteins as Cd14, which is considered to bind with LPS (lipopolysaccharide) of Gram-negative bacteria to serve as a cofactor of TLR4, thereby suppress IgE and exhibit an antiallergic action, and Tlr3, which is mainly expressed in cells responsible for natural immunity such as dendritic cells and macrophages, and recognizes double stranded RNAs derived from viruses to function for the defense against infection. It is considered that increase of expression of Cd14 contributes to suppression of allergy.

On the other hand, the mRNAs of which expression amounts decreased included those derived for genes coding for Cc127, which is known to increase in serum of atopic dermatitis patients, and Gata3, which is a Th2 cell-specific transcription factor. It is considered that decrease of expression of such genes contributes to suppression of allergy.

The Bifidobacterium longum BB536 is known to have an antiallergic action, and it has been suggested that this action is not based on enhancement of the Th1 reaction like lactic acid bacteria, but based on suppression of the Th2 reaction (Takahashi, N. et al., Clin. Exp. Immunol., 145:130-138, 2006). It is said that neonates are generally in a Th2-dominant state, and it is considered that continuous bias of Th1/Th2 balance to the Th2 side is the cause of onset of allergy. Therefore, it is considered that such suppression of the Th2 reaction as mentioned above promotes normal development of immunity in infants.

In addition, many mRNAs derived from genes coding for proteins that promote growth of children (baby mammals), for example, hormones such as growth hormone, cholecystokinin and gastrin, growth factors such as epidermal growth factor, fibroblast growth factor, platelet-derived growth factor, and vascular endothelial growth factor, enzymes such as fatty acid synthase, lipoprotein lipase, and acyl-CoA synthase, and so forth were also contained.

### INDUSTRIAL APPLICABILITY

According to the present invention, a diet or a component contained therein that provides production of milk having an immunoregulatory action can be screened for. Further, a method for producing a dairy product having an immunoregulatory action is provided by the present invention. The composition for oral ingestion of the present invention has an immunostimulating action or an immunosuppressive action, and is useful especially for infants.

1. A method for screening for a diet or a substance providing production of breast milk having an immunoregulatory action, which comprises identifying a diet or a substance that increases or decreases an amount of mRNA present in milk of a mammal by correlating a mRNA profile in the milk and an index selected from the group consisting of a diet ingested by the mammal and a substance contained in the diet.

2. The method according to claim 1, wherein the immunoregulatory action is an immunostimulating action, and when either one or both of the following conditions are satisfied, it is determined that the diet or the substance provides production of breast milk having an immunostimulating action:
(A) the mRNA is a mRNA of which an amount in milk of a rat increases when a Bifidobacterium is orally administered to the rat, and ingestion of the diet increases the amount of the mRNA,
(B) the mRNA is a mRNA of which an amount in milk of a rat decreases when a Bifidobacterium is orally administered to the rat, and ingestion of the diet decreases the amount of the mRNA.

3. The method according to claim 1, wherein the immunoregulatory action is an immunosuppressive action, and when either one or both of the following conditions are satisfied, it is determined that the diet or the substance provides production of breast milk having an immunosuppressive action:
(a) the mRNA is a mRNA of which an amount in milk of a rat increases when a Bifidobacterium is orally administered to the rat, and ingestion of the diet decreases the amount of the mRNA,
(b) the mRNA is a mRNA of which an amount in milk of a rat decreases when a Bifidobacterium is orally administered to the rat, and ingestion of the diet increases the amount of the mRNA.

4. The method according to claim 1, further comprising comparing mRNA profiles in the milk observed before and after ingestion of the diet, and determining that the diet increases the amount of the mRNA in the milk when an amount of at least one kind of mRNA in the milk observed after the ingestion is higher than that observed before the ingestion.

5. The method according to claim 2, further comprising comparing mRNA profiles in the milk observed before and after ingestion of the diet, and determining that the diet increases the amount of the mRNA in the milk when an amount of a mRNA in the milk observed after the ingestion is 1.5 times or more of that observed before the ingestion.

6. The method according to claim 1, further comprising comparing mRNA profiles in the milk observed before and after ingestion of the diet, and determining that the diet decreases the amount of the mRNA in the milk when an amount of at least one kind of mRNA in the milk observed after the ingestion is lower than that observed before the ingestion.

7. The method according to claim 2, wherein mRNA profiles in the milk observed before and after ingestion of the diet are compared, and determining that the diet decreases the amount of the mRNA in the milk when an amount of a mRNA in the milk observed after the ingestion is 0.67 times or less of that observed before the ingestion.

8. The method according to claim 1, wherein the mammal is a human, a rat, or a bovine.

9. The method according to claim 1, wherein the mRNA profiles comprises an amount of mRNA selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl2l1, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp2l1, Nr3c2, Klf4, Dhx8, Myh14, Mcam, Dusp3, Acer2, L3 mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4bp1, Naaa, Expi, Fgf7, RGD1562492, Lama2, Ahnak, Sec3l1, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdspl, Tp53bp1, Fam60a, Pik3r2, RGD1305089, Arhgef5, Nek7, Rp2, Vhl, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, Klf11, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnupll, LOC687994, Sptbn1, Dtx31, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3c11, LOC100302372, RGD1311892, Fbxo42, Daam1, K1h15, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, Slc25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, No1c1, Sult1a1, Ankrd17, Trim2, Nfib, Cd14, S100a10, LOC687105, Ccdc123, RGD1309537, Pols, Aplf, Gulp1, Slc40a1, Tfdp2, Dyrk2, Anxa5, Nr3c1, RGD1306862, Rkhd2, Cdc42bpb, Numa1, Mocos, Math, Akap11, Diaph1, Znrf1, Sos1, Rhob, Pkp2, Srf, RGD1310159, Cdh1, Rnf19a, Pdlim1, Csnk1g3, Hectd1, Taf5l, Noc3l, Nfat5, RGD1562529, Atl3, Ptpn9, Nedd4l, Wipi1, Gna1, Tle1, Pprc1, Src, Clic1, Secisbp21, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc215, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, K1h124, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbxl8, Chd1, Tp53 bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rp18, Prdx1, Phrf1, Pcm1, Mapk12, Slc1a4, Prl3d4, Trim16, LOC689842, Cacna1e, RT1-EC2, Gal, Arnt2, Stk11ip, Sdc3, RGD1563155, Tp53i11, Mpz, Dnmt31, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Ttl, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb311, Klk12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fhl3, Olr515, Tpsab1, RGD1564961, Hdhd2, Farp1, Ptprs, Hand1, Rp137a, Tcf15, Neurod2, Usp20, Foxo3, Aldh112, RGD1308706, Nov, RGD1566130, Slc34a2, Gzma, Hist1h4b, Iqgap3, Enpp2, Tm7sf2, Exo1, Slc9a1, Rp137, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, Slc25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad541, Vps37c, Hmga1, Olr1572, Furin, RT1-CE5, Rab15, Folr1, Fundc1, Pak3, Aqp2, Hapln2, Tmem37, Gjd3, Lamb1, Cox5a, Cfd, Jak3, Magt1, Sc4 mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Msl3l2, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bcan, Foxc2, Jph1, Cc127, Pdia3, Gabrq, Surf6, Runx3, Abcb6, Cdk2, LOC687813, and Porcn.

10. The method according to claim 2, wherein the mRNA of which the amount in milk of a rat increases when a Bifidobacterium is orally administered to the rat is selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl2l1, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp2l1, Nr3c2, Klf4, Dhx8, Myh14, Mcam, Dusp3, Acer2, L3 mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4bp1, Naaa, Expi, Fgf7, RGD1562492, Lama2, Ahnak, Sec3l1, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdsp1, Tp53 bp1, Fam60a, Pik3r2, RGD1305089, Arhgef5, Nek7, Rpt, Vhf, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, Klf11, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnrp11, LOC687994, Sptbn1, Dtx31, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3c11, LOC100302372, RGD1311892, Fbxo42, Daam1, Klhl5, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, Slc25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, Nolc1, Sult1a1, Ankrd17, Trim2, Nfib, Cd14, S100a10, LOC687105, Ccdc123, RGD1309537, Pols, Ap1f, Gulp1, Slc40a1, Tfdp2, Dyrk2, Anxa5, Nr3c1, RGD1306862, Rkhd2, Cdc42 bpb, Numa1, Mocos, Mafb, Akap11, Diaph1, Znrf1, Sos1, Rhob, Pkp2, Srf, RGD1310159, Cdh1, Rnf19a, Pdlim1, Csnk1g3, Hectd1, Taf5l, Noc3l, Nfat5, RGD1562529, Atl3, Ptpn9, Nedd41, Wipi1, Gnal, Tle1, Pprc1, Src, Clic1, Secisbp21, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc215, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, Klhl24, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbxl8, Chd1, Tp53 bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rp18, Prdx1, Phrf1, Pcm1, and Mapk12.

11. The method according to claim 2, wherein the mRNA of which the amount in milk of a rat decreases when a Bifidobacterium is orally administered to the rat is selected from the group consisting of Slc1a4, Prl3d4, Trim16, LOC689842, Cacna1e, RT1-EC2, Gal, Arnt2, Stk11ip, Sdc3, RGD1563155, Tp53i11, Mpz, Dnmt31, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Ttl, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb311, Klk12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fhl3, Olr515, Tpsab1, RGD1564961, Hdhd2, Farp1, Ptprs, Hand1, Rp137a, Tcf15, Neurod2, Usp20, Foxo3, Aldh112, RGD1308706, Nov, RGD1566130, Slc34a2, Gzma, Hist1h4b, Iqgap3, Enpp2, Tm7sf2, Exo1, Slc9a1, Rp137, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, S1c25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad541, Vps37c, Hmga1, Olr1572, Furin, RT1-CE5, Rab15, Folr1, Fundc1, Pak3, Aqp2, Hapln2, Tmem37, Gjd3, Lamb1, Cox5a, Cfd, Jak3, Magt1, Sc4mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Msl3l2, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bcan, Foxc2, Jph1, Cc127, Pdia3, Gabrq, Surf6, Runx3, Abcb6, Cdk2, LOC687813, and Porcn.

12. A method for producing milk or a dairy product having an immunoregulatory action, comprising the step of providing a diet or a substance identified to increase or decrease amount of mRNA in milk of a mammal by the screening method according to claim 1 to a non-human mammal, and the step of collecting milk of the mammal.

13. The method according to claim 12, wherein the immunoregulatory action is an immunostimulating action, and the diet or the substance is one determined to provide production of breast milk having an immunostimulating action.

14. The method according to claim 12, wherein the immunoregulatory action is an immunosuppressive action, and the diet or the substance is one determined to provide production of breast milk having an immunosuppressive action.

15. A method for producing a composition for oral ingestion having an immunostimulating action, comprising the step of adding a mRNA of which an amount of the mRNA in milk of a rat increases when a Bifidobacterium is orally administered to the rat to a base for a composition for oral ingestion.

16. The method according to claim 15, wherein the mRNA is selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl2l1, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp211, Nr3c2, Klf4, Dhx8, Myh14, Mcam, Dusp3, Acer2, L3 mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4bp1, Naaa, Expi, Fgf7, RGD1562492, Lama2, Ahnak, Sec3l1, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdsp1, Tp53bp1, Fam60a, Pik3r2, RGD1305089, Arhgef5, Nek7, Rp2, Vhl, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, Klf11, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnrpll, LOC687994, Sptbn1, Dtx3l, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3c11, LOC100302372, RGD1311892, Fbxo42, Daam1, Klhl5, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, S1c25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, Nolc1, Sult1a1, Ankrd17, Trim2, Nfib, Cd14, S100a10, LOC687105, Ccdc123, RGD1309537, Pols, Aplf, Gulp1, Slc40a1, Tfdp2, Dyrk2, Anxa5, Nr3c1, RGD1306862, Rkhd2, Cdc42bpb, Numa1, Mocos, Math, Akap11, Diaph1, Znrf1, Sos1, Rhob, Pkp2, Srf, RGD1310159, Cdh1, Rnf19a, Pdlim1, Csnk1g3, Hectd1, Taf5l, Noc3l, Nfat5, RGD1562529, Atl3, Ptpn9, Nedd41, Wipi1, Gnal, Tle1, Pprc1, Src, Clic1, Secisbp21, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc215, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, Klh124, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbxl8, Chd1, Tp53 bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rp18, Prdx1, Phrf1, Pcm1, and Mapk12.

17. A method for producing a composition for oral ingestion having an immunosuppressive action, comprising the step of adding a mRNA of which an amount of the mRNA in milk of a rat decreases when a Bifidobacterium is orally administered to the rat to a base for a composition for oral ingestion.

18. The method according to claim 17, wherein the mRNA is selected from the group consisting of Slc1a4, Prl3d4, Trim16, LOC689842, Cacna1e, RT1-EC2, Gal, Arnt2, Stk11ip, Sdc3, RGD1563155, Tp53i11, Mpz, Dnmt31, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Tt1, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb311, Klk12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fhl3, Olr515, Tpsab1, RGD1564961, Hdhd2, Farp1, Ptprs, Hand1, Rp137a, Tcf15, Neurod2, Usp20, Foxo3, Aldh112, RGD1308706, Nov, RGD1566130, Slc34a2, Gzma, Hist1h4b, Iqgap3, Enpp2, Tm7sf2, Exo1, Slc9a1, Rp137, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, Slc25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad541, Vps37c, Hmga1, Olr1572, Furin, RT1-CE5, Rab15, Folr1, Fundc1, Pak3, Aqp2, Hapln2, Tmem37, Gjd3, Lamb1, Cox5a, Cfd, Jak3, Magt1, Sc4mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Msl3l2, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bcan, Foxc2, Jph1, Cc127, Pdia3, Gabrq, Surf6, Runx3, Abcb6, Cdk2, LOC687813, and Porcn.

19. The method according to claim 15, wherein the composition for oral ingestion is a food for infants or a food for little children.

20. The method according to claim 19, wherein the food for infants or the food for little children is infant formula or follow-on formula.

21. The method according to claim 3, further comprising comparing mRNA profiles in the milk observed before and after ingestion of the diet, and determining that the diet increases the amount of the mRNA in the milk when an amount of a mRNA in the milk observed after the ingestion is 1.5 times or more of that observed before the ingestion.

22. The method according to claim 3, wherein mRNA profiles in the milk observed before and after ingestion of the diet are compared, and determining that the diet decreases the amount of the mRNA in the milk when an amount of a mRNA in the milk observed after the ingestion is 0.67 times or less of that observed before the ingestion.

23. The method according to claim 6, wherein mRNA profiles in the milk observed before and after ingestion of the diet are compared, and determining that the diet decreases the amount of the mRNA in the milk when an amount of a mRNA in the milk observed after the ingestion is 0.67 times or less of that observed before the ingestion.

24. The method according to claim 3, wherein the mRNA of which the amount in milk of a rat increases when a Bifidobacterium is orally administered to the rat is selected from the group consisting of Cyp24a1, Dnm1, Dpysl2, Fam89a, Arhgap22, Fbxl5, LOC304396, Btg2, Csnk1g1, Mll1, Eml4, Nelf, Aldh1l1, Boc, Usp43_predicted, Rassf5, Man2a1, Srgn, Gbf1, RGD1562551, Lrba, P2ry2, Bcl2l1, Rbm14, RGD1307365, Tecpr1, Kctd9, Pkig, RGD1308139, Klhl35, Gab1, Arid5b, Zfp157, Mafk, Trim47, Ddx21, Tiparp, Hspa1b, Tcfcp2l1, Nr3c2, Klf4, Dhx8, Myh14, Mcam, Dusp3, Acer2, L3 mbtl3, Fcho2, RT1-N3, Ash1l, Paxip1, Irgq, Plcb1, Sts, Spetex-2D, Zmym3, Anxa1, Rtn4ip1, Gngt1, Srcap, Otud4, RGD1307569, Adpgk, Plk3, Phf1, St14, N4bp1, Naaa, Expi, Fgf7, RGD1562492, Lama2, Ahnak, Sec3l1, Mks1, Prodh, Baz1b, R3hdm1, Setd5, Rhebl1, Kdm5b, Fbxw8, Epb4.1l4a, Lpin2, Cxcl2, Nr2c2, Ckap4, Ankrd1, Ugcg, Fcgr2b, Sf3a1, Parg, Heatr6, Ctdsp1, Tp53bp1, Fam60a, Pik3r2, RGD1305089, Arhgef5, Nek7, Rpt, Vhl, Usp31, Camsap1, RGD1308759, Ldlrap1, RGD1359108, Sp1, Mast4, Rela, Nhlrc3, Intu, Gcap14, Spetex-2B, Cdc42se2, Chd7, Rbms1, Invs, LOC100294508, Kif2a, Exoc6, Zbtb1, Rnd3, Rb1cc1, Sphk1, Ugt1a2, Noxo1, Mllt6, Thumpd2, Ptpn4, Thex1, S100a8, Grk5, Trove2, Pcdh17, Med13, LOC684871, RGD1305350, Fam82a2, Rab43, Clip1, Ankrd57, RGD1563375, Crim1, Celsr2, Idh1, Lrrc61, Aff4, Arhgap1, Itpr3, Lhfp, Dnajc12, Garnl1, Gtf3c5, Hspb8, Chac1, Pof1b, Ptpre, Arl6ip1, Son, Ppargc1b, Med1, Casp12, Zbtb39, Urb1, Wdr89, Josd1, Pank4, Ska2, Rrm1, K1f11, Phc1, Birc6, Cask, Mesdc2, St5, Smyd5, Heca, Plagl2, Strn4, Ftsjd1, Maml1, RGD1561817, Anxa8, Nup153, Anxa2, Trappc2, Gpr155, Zfhx3, Morc2, Lrsam1, Polg, RGD1311307, Kcnk4, Tmcc3, RGD1562136, Pde4b, Lkap, Chn1, Dusp16, Pex5, Ns5atp9, Map6, Angptl4, Dnmt3b, Fert2, Gzf1, Plcb4, Ap4m1, Stat3, Rkhd1, LOC691223, Dcp2, Rtn4, Tlr3, Tubb6, Casp8, Nin, Fyn, Tmem77, Maml3, Slc15a4, Sqrdl, Ppp1r9b, Pafah1b1, Def8, Fam164a, Znf746, Bmyc, RGD1305235, Thada, Hip1, Pgrmc2, Sgk3, Baz1a, Foxn3, Map3k12, Efhd2, Pogz, Pcf11, RGD1560523, Mcart1, Sox4, Calcoco1, Myom1, Mtmr9, Akap13, Rhobtb1, Matr3, LOC684297, Rxrb, Mars2, Abcb8, Bik, Med22, Cryab, Cpt1a, Cnksr1, Kirrel3, Arl13a, Sept8, Rb1, Prmt2, Ccnb3, Zhx2, Pds5b, Hdac6, Prdm4, Smarcad1, Usp9x, Sdc1, Arhgap28, Rad17, Sorbs1, Tp63, Wdr48, Hnrpll, LOC687994, Sptbn1, Dtx31, Cnnm2, Fbln5, Usp48, Tgm2, RGD1564776, RGD1563106, Taok1, Snx30, Utp6, Cytip, Rest, Eif4g3, Adnp2, Wdr1, Hiat1, Ddx58, RGD1359600, Arhgap12, Dapk2, Nup160, Eea1, LOC363188, RGD1563437, Paqr5, LOC500684, Ddhd1, Kdm6b, Ube2d1, Rhoc, LOC361100, Cx3c11, LOC100302372, RGD1311892, Fbxo42, Daam1, Klhl5, Fbxo30, Syt12, Herc1, Dab2ip, Tacc2, Sipa1l1, Ets2, Zbtb43, Fhdc1, Pdlim5, Stxbp5, Hdac8, Sestd1, Acot2, Fam117b, Nrip1, Aldh3a2, Exosc8, Shmt1, Lrrc57, Rell2, Zfyve16, Cebpd, Tnrc6b, Nsun6, Dennd5a, Fbxw5, Slc25a25, Dtwd1, Hspb1, Homer2, Nhej1, Etnk1, Nol9, Med23, Nup133, Rnd1, Rplp1, Ankfy1, Pik3r4, Plcd1, Cxcl12, Lix1l, Ralbp1, Cald1, LOC302495, Cdcp1, Ralb, Pcyt1a, Skil, Trim25, Pigv, Mapk3, Pard6g, Klf7, Pxn, Dnaja4, Cnot1, Capn2, Birc3, Frmd8, Chd2, Prpf39, Taf5, Eif4enif1, Pkn2, Fam179b, Kdm2a, Gda, Ccdc21, Il17re, Eif4e3, Dock8, Nolc1, Sult1a1, Ankrd17, Trim2, Nfib, Cd14, S100a10, LOC687105, Ccdc123, RGD1309537, Pols, Aplf, Gulp1, Slc40a1, Tfdp2, Dyrk2, Anxa5, Nr3c1, RGD1306862, Rkhd2, Cdc42bpb, Numa1, Mocos, Math, Akap11, Diaph1, Znrf1, Sos1, Rhob, Pkp2, Srf, RGD1310159, Cdh1, Rnf19a, Pdlim1, Csnk1g3, Hectd1, Taf5l, Noc3l, Nfat5, RGD1562529, Atl3, Ptpn9, Nedd4l, Wipi1, Gnal, Tle1, Pprc1, Src, Clic1, Secisbp2l, Rnf103, Zfp772, Ogt, Zcchc2, Clasp1, Gtpbp2, Ift172, LOC500625, RGD1309350, Map3k6, Plce1, Nedd4, Cpne3, Prim2, Trafd1, Myef2, Znf532, Tmem50b, Il6ra, Pdcd6ip, LOC500757, Tpm4, Rnasen, Lpin1, Flot1, Cds1, Tnfaip1, Mudeng, Cdc215, Arid2, Znf654, Nat8b, Snx12, Ap1g1, Itsn2, Fam21c, RGD1306820, Mesdc1, Tmco6, Fkbp5, Prpf8, Pdcd11, Runx1, Epn2, Arid4b, Slk, Prkx, Dnajb3, Phf17, Fam193a, Pvrl2, Tep1, Atxn7, Wwtr1, LOC313672, Mus81, C1d, Arid4a, Ehd1, Usp32, Carhsp1, Acss1, Ctcf, Bag3, Mtrr, Ywhah, Csrp1, Smc3, Sorbs2, RGD1309762, Ptpn1, Setd2, Efr3a, Tnk1, Ncor1, Coq4, Klh124, Cox11, Tfip11, Rcan1, Nagk, Ep300, Myst3, Rbm28, Anapc7, Ezh1, Cyp27a1, Irs2, Sdccag1, Foxp1, Dusp18, Hcca2, Fbxl8, Chd1, Tp53bp2, RGD1309995, RGD1310552, Kif5b, Pcaf, Slc25a42, Ocln, RGD1559961, Kifap3, Wtip, Gapvd1, Ankrd42, Supt4h1, Haus1, RGD1310951, Prdm2, Arvcf, Gtf2h1, Rassf6, Pcif1, Arap2, Ube2k, Twf1, Rp18, Prdx1, Phrf1, Pcm1, and Mapk12.

25. The method according to claim 3, wherein the mRNA of which the amount in milk of a rat decreases when a Bifidobacterium is orally administered to the rat is selected from the group consisting of Slc1a4, Prl3d4, Trim16, LOC689842, Cacna1e, RT1-EC2, Gal, Arnt2, Stk11ip, Sdc3, RGD1563155, Tp53i11, Mpz, Dnmt31, Ache, Cln5, Olr184, Asb7, Rtp3, Peli2, Caskin2, Cass4, Plvap, LOC680711, Nat2, RGD1565498, Hist1h1a, Col22a1, LOC100360528, Htr6, Trim42, Ccdc89, Atp10d, Klri2, LOC100360801, RGD1560019, Ugt2b37, Oprk1, Cyct, Pdzd2, Vom2r31, Sh2d5, Tmem136, Gh1, Tt1, Pgbd5, Zfp583, Ankrd2, Avil, Mcm3ap, Mypn, Frmd4a, Rnf182, Kcng1, Paqr7, RGD1304929, RGD1565100, Pkp1, Nnmt, Csrnp1, Siae, Hoxb6, Pga5, Hoxd9, RGD1308541, Cyth3, Fbp1, Bnc1, Col18a1, Traf3, Sema7a, Rcn3, Ift122, Olr1275, LOC619574, Slc5a4a, Smc2, Cldn18, LOC679462, Pogk, Olr445, Abhd15, LOC688219, RGD1564846, Pias4, Alg8, Nkx2-4, Mmp2, Iqcf1, B9d2, Fam107a, Rarres1, RGD1561276, Wbp2, Wdr93, C1qtnf5, Olr1303, Egfl7, Olr507, Slc32a1, RGD1563516, Id3, LOC691130, Ccdc58, Sox21, Tnfsf12, Qrich2, RGD1308930, LOC307727, B3galnt2, Tob2, Creb311, Klk12, Dqx1, Ncoa2, Slfnl1, Elf2, Gsx2, Hhip, Grm2, Fam170a, Pnma3, Gpc2, Npvf, RGD1308143, Tsen54, Igfbp1, Zdhhc19, Csrnp2, Dcst2, Krt1, Nanos1, Cdh13, Ebi3, Prr3, Rnf43, Btbd17, Olr1626, Trim26, Gata3, Cdkn1c, Hrc, Grasp, Galm, RGD1308106, Samd14, Olr471, Oprd1, Pim1, Il2rb, Ptpn13, Mtus1, RGD1305298, Fhl3, Olr515, Tpsab1, RGD1564961, Hdhd2, Farp1, Ptprs, Hand1, Rp137a, Tcf15, Neurod2, Usp20, Foxo3, Aldh112, RGD1308706, Nov, RGD1566130, Slc34a2, Gzma, Hist1h4b, Iqgap3, Enpp2, Tm7sf2, Exo1, Slc9a1, Rp137, RGD1565970, Pde12, Eefsec, Dmrta2, LOC498685, Brunol5, Ocm, Olr4, Tmem164, RGD1561843, Flad1, Frmd4b, Marcksl1, Bhlhe22, Yif1b, Ttc38, Phyh, Mfsd6, LOC257642, Fam189b, Sned1, LOC100362849, Cd3d, Slc25a29, Cd7, RGD1563281, Grb7, Ccdc147, Stat2, LOC688228, Ppp3ca, LOC688241, Shisa4, Atp8b1, Krtap1-5, Cd3g, Txnl4b, Ranbp10, Dynlt3, Atp2b2, Trub1, Zhx3, Rexo4, Snapc2, LOC685031, Rad541, Vps37c, Hmga1, Olr1572, Furin, RT1-CE5, Rab15, Folr1, Fundc1, Pak3, A 2, Hapln2, Tmem37, Gjd3, Lamb1, Cox5a, Cfd, Jak3, Magt1, Sc4mol, Uros, Calca, Abca7, Pm20d2, Dpf2, Tmprss4, Znf467, Btbd9, Rasgrf1, RGD1310270, Adam15, Kif15, Plekho2, Atp2c2, Rps25, Lipa, Cpeb2, Znf575, LOC287010, Arhgap26, LOC311352, Znrf4, Msh5, Tmed7, Gpd1l, Ankrd37, Cox4i2, Ms1312, Snrnp70, Rps6ka5, Tomm20, Rassf4, Hes1, Ctdsp1, Pomc, Inpp4b, Nfix, Ydjc, Cst3, Tesk2, Tcn2, RGD1309847, Artn, Wibg, Prpf4, LOC685179, Igsf3, Got1, Lcp2, Agrn, Cited2, Bat4, RGD1308059, Sft2d1, Ubac2, Scly, Wrb, Zfp161, Fads2, Fbxo8, RGD1563148, Vegfb, Lpl, Chaf1a, Srrd, Steap1, Fkbp11, Slc39a3, Zfp53, Bean, Foxc2, Jph1, Cc127, Pdia3, Gabrq, Surf6, Runx3, Abcb6, Cdk2, LOC687813, and Porcn.

26. The method according to claim 17, wherein the composition for oral ingestion is a food for infants or a food for little children.

27. The method according to claim 26, wherein the food for infants or the food for little children is infant formula or follow-on formula.
